(19) United States
(12) Patent Application Publication
(10) Pub. No.: US 2025/0339315 A1
(43) Pub. Date: Nov. 6, 2025

(54) DOSE DOCK FOR SYRINGE

(71) Applicant: Genentech, Inc., South San Francisco, CA (US)

(72) Inventors: Thomas E. Meyer, Philadelphia, PA (US); Chase Wooley, Floyds Knobs, IN (US); Kirsten Stoner, King of Prussia, PA (US); Justin Creel, King of Prussia, PA (US); Michael Keane, Downingtown, PA (US); Sergio Camacho, King of Prussia, PA (US); Mark Hedgeland, King of Prussia, PA (US); John Miser, Sellersburg, IN (US); Rory Klingensmith, Philadelphia, PA (US); Peter Meinz, Minneapolis, MN (US)

(21) Appl. No.: 19/266,476

(22) Filed: Jul. 11, 2025

Related U.S. Application Data
(63) Continuation of application No. PCT/IB2024/000027, filed on Jan. 10, 2024.
(60) Provisional application No. 63/438,929, filed on Jan. 13, 2023.

(51) Int. Cl. A61F 9/00

(52) U.S. Cl. CPC A61F 9/0017

(57) ABSTRACT

An apparatus includes a body, a flexible cannula, and a needle. The needle is resiliently biased to extend along at least one proximal curve through least one proximal curved needle portion. The needle is resiliently biased to extend along at least one distal curve through at least one distal curved needle portion. In another embodiment, an apparatus includes a main body portion including a proximal channel configured to receive a plunger of a syringe, and a stop surface configured to selectively engage the plunger of the syringe to arrest distal advancement of the plunger. The apparatus also includes first and second deflectable beams configured to cooperate with each other to securely grip the barrel of the syringe, and first and second deflectable flanges configured to cooperate with each other to grip the barrel of the syringe.

### PRIORITY

This application claims the benefit of U.S. Pat. App. No. 63/438,929, entitled “Dose Dock for Syringe,” filed Jan. 13, 2023, the disclosure of which is incorporated by reference herein.

### BACKGROUND

The human eye comprises several layers. The white outer layer is the sclera, which surrounds the choroid layer. The retina is interior to the choroid layer. The sclera contains collagen and elastic fiber, providing protection to the choroid and retina. The choroid layer includes vasculature providing oxygen and nourishment to the retina. The retina comprises light sensitive tissue, including rods and cones. The macula is located at the center of the retina at the back of the eye, generally centered on an axis passing through the centers of the lens and cornea of the eye (i.e., the optic axis). The macula provides central vision, particularly through cone cells.

Macular degeneration is a medical condition that affects the macula, such that people suffering from macular degeneration may experience lost or degraded central vision while retaining some degree of peripheral vision. Macular degeneration may be caused by various factors such as age (also known as “AMD”) and genetics. Macular degeneration may occur in a “dry” (nonexudative) form, where cellular debris known as drusen accumulates between the retina and the choroid, resulting in an area of geographic atrophy. Macular degeneration may also occur in a “wet” (exudative) form, where blood vessels grow up from the choroid behind the retina. Even though people having macular degeneration may retain some degree of peripheral vision, the loss of central vision may have a significant negative impact on the quality of life. Moreover, the quality of the remaining peripheral vision may be degraded and, in some cases, may disappear as well. It may therefore be desirable to provide treatment for macular degeneration to prevent or reverse the loss of vision caused by macular degeneration. In some cases, it may be desirable to provide such treatment in a highly localized fashion, such as by delivering a therapeutic substance in the subretinal layer (under the neurosensory layer of the retina and above the retinal pigment epithelium) directly adjacent to the area of geographic atrophy, near the macula. However, since the macula is at the back of the eye and underneath the delicate layer of the retina, it may be difficult to access the macula in a practical fashion.

While a variety of surgical methods and instruments have been made and used to treat an eye, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a perspective view of an example of an instrument for subretinal administration of a therapeutic agent from a suprachoroidal approach;

FIG. 2 depicts a perspective view of a distal portion of a cannula of the instrument of FIG. 1;

FIG. 3 depicts a front elevation view of the distal portion of the cannula of FIG. 2;

FIG. 4 depicts a top plan view of the distal portion of the cannula of FIG. 2;

FIG. 5 depicts a perspective view of the distal end of a cannula of FIG. 2, with a needle extending from the cannula;

FIG. 6 depicts a perspective view of the instrument of FIG. 1, mounted near a patient, with a combination of medical equipment;

FIG. 7A depicts a cross-sectional side view of an eye of a patient;

FIG. 7B depicts a cross-sectional side view of the eye of FIG. 7A, with a suture loop attached to the eye, and with a sclerotomy being performed;

FIG. 7C depicts a cross-sectional side view of the eye of FIG. 7A, with the cannula of FIG. 2 being inserted through the sclerotomy opening and in between the sclera and choroid of the eye;

FIG. 7D depicts a cross-sectional side view of the eye of FIG. 7A, with the distal end of the cannula being positioned adjacent to a target location;

FIG. 7E depicts a cross-sectional side view of the eye of FIG. 7A, with the needle of FIG. 5 being advanced through the choroid to access the subretinal space at the target location;

FIG. 7F depicts a cross-sectional side view of the eye of FIG. 7A, with the needle of FIG. 5 dispensing a first volume of leading bleb fluid to provide separation between a region of the retina and the choroid at the target location;

FIG. 7G depicts a cross-sectional side view of the eye of FIG. 7A, with the needle of FIG. 5 dispensing a therapeutic agent between a region of the retina and the choroid at the target location;

FIG. 8 depicts a perspective view of an alternative cannula that may be incorporated into the instrument of FIG. 1, the cannula having a varying stiffness along a length of the cannula;

FIG. 9 depicts a front elevation view of the cannula of FIG. 8;

FIG. 10 depicts a side elevation view of the cannula of FIG. 8;

FIG. 11 depicts a top plan view of the cannula of FIG. 8;

FIG. 12 depicts a cross-sectional end view of a medial segment of the cannula of FIG. 8, taken along line 12-12 of FIG. 11;

FIG. 13 depicts a cross-sectional end view of a distal segment of the cannula of FIG. 8, taken along line 13-13 of FIG. 11;

FIG. 14 depicts a side elevation view of an alternative needle that may be incorporated into the instrument of FIG. 1, the needle having a proximal curved portion and a distal curved portion;

FIG. 15 depicts a side elevation view of a distal end of the needle of FIG. 14;

FIG. 16 depicts a side elevation view of another alternative needle that may be incorporated into the instrument of FIG. 1, the needle having a proximal curved portion and a distal curved portion;

FIG. 17 depicts a side elevation view of a distal end of the needle of FIG. 16;

FIG. 18 depicts a side elevation view of another alternative needle that may be incorporated into the instrument of FIG. 1, the needle having a proximal curved portion and a distal curved portion;

FIG. 19 depicts a side elevation view of a distal end of the needle of FIG. 18;

FIG. 20 depicts a side elevation view of another alternative needle that may be incorporated into the instrument of FIG. 1, the needle having first and second proximal curved portions and a distal curved portion;

FIG. 21 depicts a side elevation view of a distal end of the needle of FIG. 20;

FIG. 22 depicts a side elevation view of the needle of FIG. 20, showing a first reference circle for the first proximal curved portion;

FIG. 23 depicts a side elevation view of the needle of FIG. 20, showing a second reference circle for the second proximal curved portion;

FIG. 24 depicts a side elevation view of another alternative needle that may be incorporated into the instrument of FIG. 1, the needle having first and second distal curved portions;

FIG. 25 depicts a side elevation view of a distal end of the needle of FIG. 24;

FIG. 26 depicts a side elevation view of the needle of FIG. 24, showing a reference circle for the first distal curved portion;

FIG. 27 depicts a side elevation view of another alternative needle that may be incorporated into the instrument of FIG. 1, the needle having a proximal curved portion and first and second distal curved portions;

FIG. 28 depicts a side elevation view of a distal end of the needle of FIG. 27;

FIG. 29 depicts a side elevation view of another alternative needle that may be incorporated into the instrument of FIG. 1, the needle having first and second proximal curved portions and first and second distal curved portions;

FIG. 30 depicts a side elevation view of a distal end of the needle of FIG. 29;

FIG. 31 depicts a perspective view of an example of dose dock for use with a syringe;

FIG. 32 depicts a top plan view of the dose dock of FIG. 31;

FIG. 33 depicts a side elevation view of the dose dock of FIG. 31;

FIG. 34 depicts a rear elevation view of the dose dock of FIG. 31;

FIG. 35A depicts a top plan view of the dose dock of FIG. 31 and a syringe, with the syringe being separated from the dose dock, and with the syringe being coupled with a fluid source;

FIG. 35B depicts a top plan view of the dose dock of FIG. 31 and the syringe of FIG. 35A, with the syringe being separated from the dose dock, and with the syringe having drawn fluid from the fluid source;

FIG. 35C depicts a top plan view of the dose dock of FIG. 31 and the syringe of FIG. 35A, with the syringe being coupled with the dose dock while holding the drawn fluid of FIG. 35B, and with a shoulder of the plunger of the syringe being proximally spaced apart from a stop surface of the dose dock;

FIG. 35D depicts a top plan view of the dose dock of FIG. 31 and the syringe of FIG. 35A, with the syringe being coupled with the dose dock while holding the drawn fluid of FIG. 35B, with the shoulder of the plunger of the syringe being proximally spaced apart from the stop surface of the dose dock, and with the syringe coupled with the instrument of FIG. 1;

FIG. 35E depicts a top plan view of the dose dock of FIG. 31 and the syringe of FIG. 35A, with the syringe being coupled with the dose dock, with the syringe coupled with the instrument of FIG. 1, and with the plunger of the syringe being advanced distally to a first longitudinal position relative to the barrel of the syringe such that the shoulder of the plunger engages the stop surface of the dose dock;

FIG. 35F depicts a top plan view of the dose dock of FIG. 31 and the syringe of FIG. 35A, with the syringe being separated from the dose dock, with the syringe coupled with the instrument of FIG. 1, and with the plunger of the syringe remaining in the first longitudinal position relative to the barrel of the syringe; and

FIG. 35G depicts a top plan view of the dose dock of FIG. 31 and the syringe of FIG. 35A, with the syringe being separated from the dose dock, with the syringe coupled with the instrument of FIG. 1, and with the plunger of the syringe being advanced distally to a second longitudinal position relative to the barrel of the syringe such that the shoulder of the plunger engages a stop surface of the barrel.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

For clarity of disclosure, the terms “proximal” and “distal” are defined herein relative to a surgeon or other operator grasping a surgical instrument having a distal surgical end effector. The term “proximal” refers the position of an element closer to the surgeon or other operator and the term “distal” refers to the position of an element closer to the surgical end effector of the surgical instrument and further away from the surgeon or other operator.

Furthermore, the terms “about,” “approximately,” and the like as used herein in connection with any numerical values or ranges of values are intended to encompass the exact value(s) referenced as well as a suitable tolerance that enables the referenced feature or combination of features to function for the intended purpose described herein.

### I. INSTRUMENT FOR SUBRETINAL ADMINISTRATION OF THERAPEUTIC AGENT

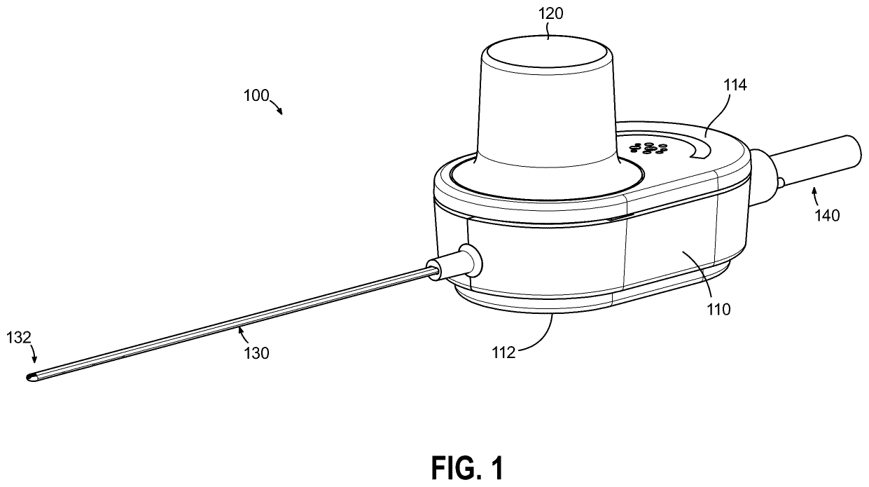

FIG. 1 shows an example of an instrument (100) that is configured for use in a procedure for the subretinal administration of a therapeutic agent to an eye of a patient from a suprachoroidal approach. Instrument (100) comprises a body (110) and a flexible cannula (130) extending distally from body (110). Cannula (130) of the present example has a generally rectangular cross section, though any other suitable cross-sectional profile (e.g., elliptical, etc.) may be used. The generally rectangular cross-sectional profile of cannula (130) is configured to enable cannula (130) to be passed atraumatically along the suprachoroidal space, as will be described in greater detail below. Cannula (130) is generally configured to support a needle (150) that is slidable within cannula (130), as will be described in greater detail below.

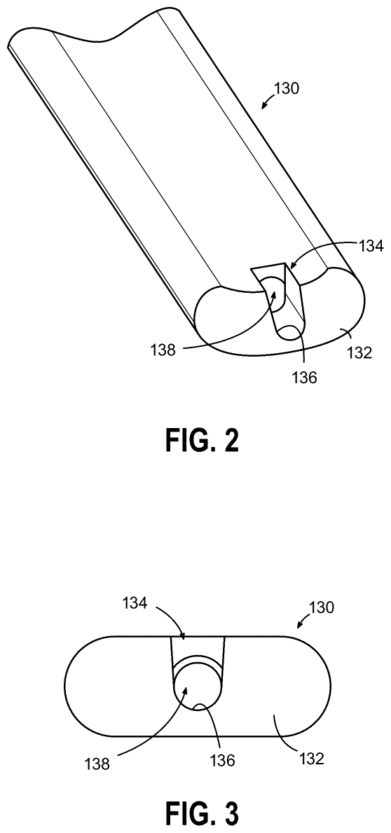

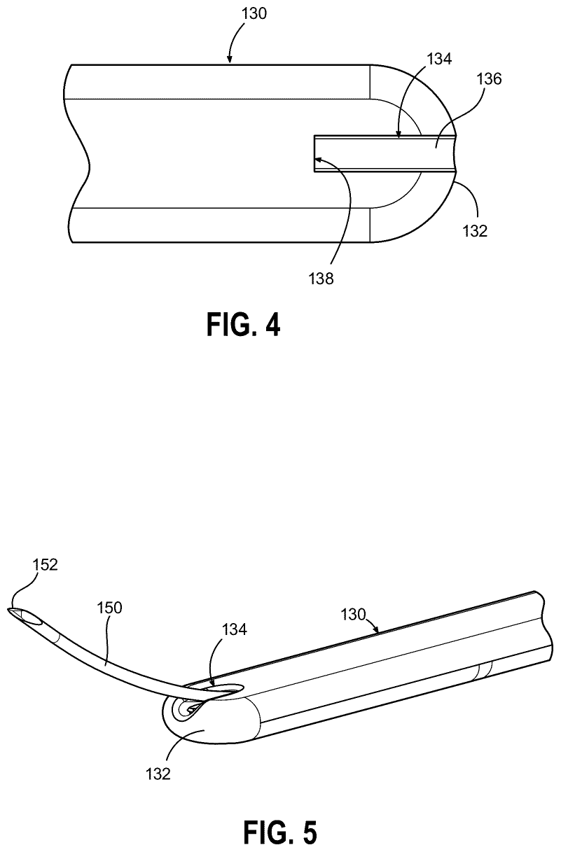

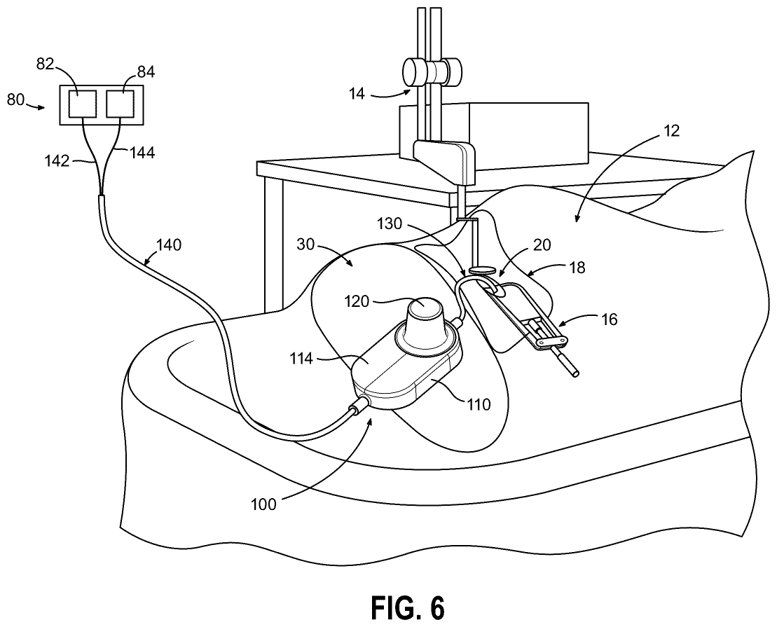

In the present example, cannula (130) comprises a flexible material such as Polyether block amide (PEBA), though any other suitable material or combination of materials may be used. In some versions, cannula (130) has a cross-sectional profile dimension of approximately 1.6 mm (width) by approximately 0.6 mm (height), with a length of approximately 80 mm. Alternatively, any other suitable dimensions may be used. Cannula (130) of the present example is flexible enough to conform to specific structures and contours of the patient's eye, yet cannula (130) has sufficient column strength to permit advancement of cannula (130) between the sclera and choroid of patient's eye without buckling. As best seen in FIGS. 2-5, cannula (130) includes a transversely oriented opening (134) near the distal end (132) of cannula (130). Opening (134) of the present example is formed by a U-shaped lateral recess (136) in cannula (130), which leads to an open distal end (138) of a needle guide lumen within cannula (130). Distal end (132) is atraumatic such that distal end (132) is configured to provide separation between the sclera and choroid layers via blunt dissection, as will be described in greater detail below, to thereby enable cannula (130) to be advanced between such layers while not inflicting trauma to the sclera or choroid layers.

By way of example only, cannula (130) may be configured and operable in accordance with at least some of the teachings of U.S. Pat. No. 10,226,379, entitled “Method and Apparatus for Subretinal Administration of Therapeutic Agent,” issued Mar. 12, 2019, the disclosure of which is incorporated by reference herein, in its entirety; U.S. Pat. No. 10,646,374, entitled “Apparatus and Method to From Entry Bleb for Subretinal Delivery of Therapeutic Agent,” issued May 12, 2020, the disclosure of which is incorporated by reference herein, in its entirety; and/or in any other suitable fashion.

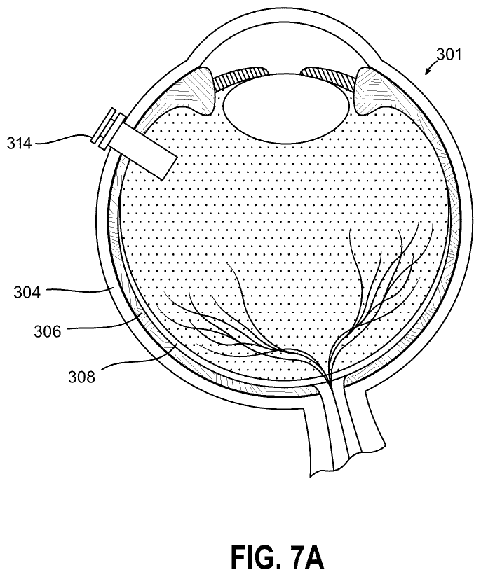

As shown in FIG. 5, needle (150) may be advanced distally to protrude from opening (134). Needle (150) of the present example has a sharp distal tip (152) and defines a lumen (not shown). Distal tip (152) of the present example has a lancet configuration. In some other versions, distal tip (152) has a tri-bevel configuration or any other configuration as described in U.S. Pat. No. 10,226,379, the disclosure of which is incorporated by reference herein, in its entirety. Still other suitable forms that distal tip (152) may take will be apparent to those skilled in the art in view of the teachings herein. In the present example, the generally rectangular, generally elliptical, or otherwise generally flat cross-sectional profile of cannula (130) prevents cannula (130) from rotating about the longitudinal axis of cannula (130) when cannula (130) is disposed in the suprachoroidal space as will be described in greater detail below. This provides a consistent and predictable orientation of opening (134), thereby providing a consistent and predictable exit path for needle (150) when needle (150) is advanced distally relative to cannula (130) as will be described in greater detail below.

By way of example only, the angle defined between the exposed portion of needle (150) and cannula (130), after needle (150) has been advanced distally relative to cannula (130), may be within the range of approximately 5° to approximately 30° relative to the longitudinal axis of cannula (130); or more particularly within the range of approximately 5° to approximately 20° relative to the longitudinal axis of cannula (130); or more particularly within the range of approximately 5° to approximately 10° relative to the longitudinal axis of cannula (130); or more particularly within the range of approximately 7° and approximately 9° relative to the longitudinal axis of cannula (130). In the present example, needle (150) is resiliently biased to assume a bent configuration to thereby provide an exit angle that varies based on the extent to which needle (150) is advanced distally relative to cannula (130). By way of further example only, needle (150) may include a preformed bend in accordance with at least some of the teachings of U.S. Pat. No. 10,478,553, entitled “Apparatus for Subretinal Administration of Therapeutic Agent via a Curved Needle,” issued Nov. 19, 2019, the disclosure of which is incorporated by reference herein, in its entirety.

As shown in FIG. 1, instrument (100) of the present example further comprises an actuation knob (120) located at a top portion (114) of body (110). Actuation knob (120) is rotatable relative to body (110) to thereby selectively translate needle (150) longitudinally relative to cannula (130). In particular, actuation knob (120) is rotatable in a first angular direction to drive needle (150) distally relative to cannula (130); and in a second angular direction to drive needle (150) proximally relative to cannula (130). By way of example only, instrument (100) may provide such functionality through knob (120) in accordance with at least some of the teachings of U.S. Pat. No. 10,646,374, the disclosure of which is incorporated by reference herein, in its entirety. Other suitable ways in which rotary motion of knob (120) may be converted to linear translation of needle (150) will be apparent to those skilled in the art in view of the teachings herein. Similarly, other suitable ways in which needle (150) may be actuated (150) longitudinally relative to cannula (130) will be apparent to those skilled in the art in view of the teachings herein. As also shown in FIG. 1, a conduit assembly (140) extends proximally from body (110). Conduit assembly (140) is configured to contain one or more fluid conduits (not shown) that are in fluid communication with needle (150). In some versions such fluid conduits are coupled with sources of leading bleb fluid and therapeutic agent.

### II. PROCEDURE FOR DELIVERY OF A THERAPEUTIC AGENT TO SUBRETINAL SPACE FROM A SUPRACHOROIDAL APPROACH

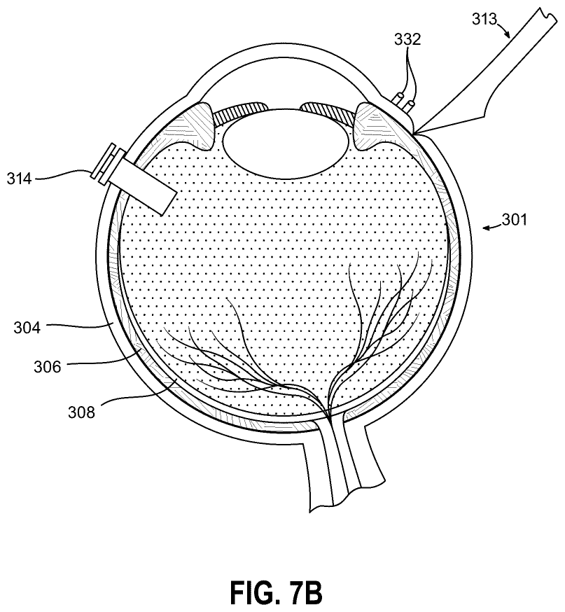

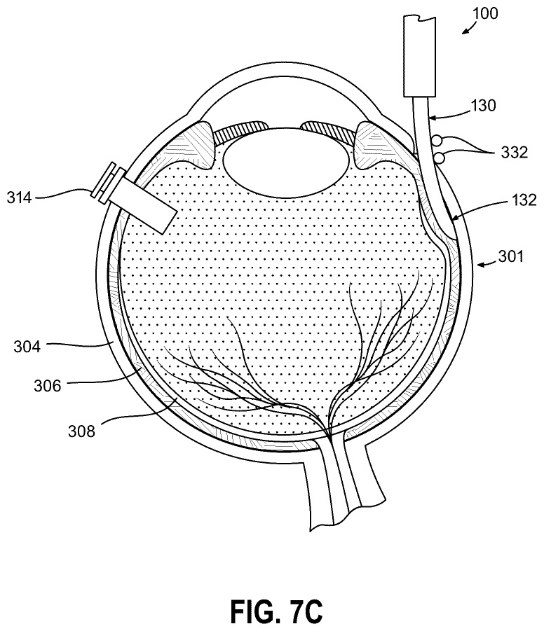

FIG. 6 shows a scenario where instrument (100) is positioned in relation to a patient. In this example, a drape (12) is disposed over the patient, with an opening (18) formed in drape (12) near the patient's eye (301). A speculum (16) is used to keep the eye (301) open. A fixture (14) is positioned adjacent to the eye (301). Fixture (14) may be used to secure instrumentation, such as a viewing scope, relative to the patient. A magnetic pad (30) is adhered to drape (12) near the opening (18) adjacent to the eye (301). Instrument (100) is placed on magnetic pad (30) and is removably secured thereto via magnetic attraction. In the present example, one or more permanent magnets (not shown) are positioned within body (110) near bottom potion (112); and these magnets are magnetically attracted to one or more ferrous elements (not shown) contained within magnetic pad (30). By way of example only, these magnets and magnetic pad (30) may be configured in accordance with at least some of the teachings of U.S. Pat. No. 10,806,629, entitled “Injection Device for Subretinal Delivery of Therapeutic Agent,” issued Oct. 20, 2020, the disclosure of which is incorporated by reference herein, in its entirety. Instrument (100) is oriented to enable insertion of flexible cannula (130) of instrument (100) into the eye (301). An example of a process for inserting and positioning cannula (130) in the eye (301) is described in greater detail below with reference to FIGS. 7A-7F.

In the present example, instrument (100) is coupled with a fluid delivery system (80) via conduit assembly (140). In this example, fluid delivery system (80) comprises a bleb fluid source (82) and a therapeutic agent fluid source (84). Bleb fluid source (82) is coupled with a bleb fluid conduit (142) of conduit assembly (140); and therapeutic agent fluid source (84) is coupled with a therapeutic agent conduit (144) of conduit assembly (140). Conduits (142, 144) are in fluid communication with needle (150). In some versions, fluid sources (82, 84) comprise syringes. In some other versions, fluid sources (82, 84) comprise separate reservoirs and one or more associated pumps and/or valves, etc.

FIGS. 7A-7G show an example of a procedure that may be carried out using the above-described equipment, to deliver a therapeutic agent to the subretinal space of the eye (301) from a suprachoroidal approach. By way of example only, the method described herein may be employed to treat macular degeneration and/or other ocular conditions. Although the procedure described herein is discussed in the context of the treatment of age-related macular degeneration, no such limitation is intended or implied. For instance, in some alternative procedures, the same techniques described herein may be used to treat retinitis pigmentosa, diabetic retinopathy, and/or other ocular conditions. Additionally, the procedure described herein may be used to treat either dry or wet age-related macular degeneration, among other conditions.

In the present example, the procedure begins by an operator immobilizing tissue surrounding a patient's eye (301) (e.g., the eyelids) using an instrument such as speculum (16), and/or any other instrument suitable for immobilization. While immobilization described herein with reference to tissue surrounding eye (301), eye (301) itself may remain free to move. Once the tissue surrounding eye (301) has been immobilized, an eye chandelier port (314) is inserted into eye (301), as shown in FIG. 7A, to provide intraocular illumination when the interior of eye (301) is viewed through the pupil. In the present example, eye chandelier port (314) is positioned in the inferior medial quadrant such that a superior temporal quadrant sclerotomy may be performed. Eye chandelier port (314) is positioned to direct light onto the interior of eye (301) to illuminate at least a portion of the retina (308) (e.g., including at least a portion of the macula). As will be understood, such illumination corresponds to an area of eye (301) that is being targeted for delivery of therapeutic agent.

In the present example, only chandelier port (314) is inserted at the stage shown in FIG. 7A, without yet inserting an optical fiber (315) into port (314). In some other versions, an optical fiber (315) may be inserted into chandelier port (314) at this stage. In either case, a microscope may optionally be utilized to visually inspect the eye to confirm proper positioning of eye chandelier port (314) relative to the target site. Although FIG. 7A shows a certain positioning of eye chandelier port (314), eye chandelier port (314) may have any other suitable positioning as will be apparent to those skilled in the art in view of the teachings herein.

Once eye chandelier port (314) has been positioned, the sclera (304) may be accessed by dissecting the conjunctiva by incising a flap in the conjunctiva and pulling the flap posteriorly. After such a dissection is completed, the exposed surface of the sclera (304) may optionally be blanched using a cautery tool to minimize bleeding. Once conjunctiva dissection is complete, the exposed surface of the sclera (304) may optionally be dried using a WECK-CEL or other suitable absorbent device.

A template may then be used to mark the eye (20), as described in U.S. Pat. No. 10,226,379, the disclosure of which is incorporated by reference herein, in its entirety; and/or U.S. Pat. No. 11,000,410, entitled “Guide Apparatus for Tangential Entry into Suprachoroidal Space,” issued May 11, 2021, the disclosure of which is incorporated by reference herein, in its entirety. The operator may then use a visual guide created using the template to attach a suture loop assembly (332) and to perform a sclerotomy, as shown in FIG. 7B, using a conventional scalpel (313) or other suitable cutting instrument. By way of example only, suture loop assembly (332) may be formed in accordance with at least some of the teachings of U.S. Pat. No. 10,226,379, the disclosure of which is incorporated by reference herein, in its entirety. Alternatively, in lieu of suture loop assembly (332), the operator may install a guide tack in accordance with at least some of the teachings of U.S. Pat. No. 11,000,410, the disclosure of which is incorporated by reference herein, in its entirety.

The sclerotomy procedure forms a small incision through sclera (304) of eye (301). The sclerotomy is performed with particular care to avoid penetration of the choroid (306). Thus, the sclerotomy procedure provides access to the space between sclera (304) and choroid (306). Once the incision is made in eye (301), a blunt dissection may optionally be performed to locally separate sclera (304) from choroid (306). Such a dissection may be performed using a small blunt elongate instrument, as will be apparent to those skilled in the art in view of the teachings herein.

With the sclerotomy procedure performed, an operator may insert cannula (130) of instrument (100) through the incision and into the space between sclera (304) and choroid (306). As can be seen in FIG. 7C, cannula (130) is directed through suture loop assembly (332) and into the incision. Suture loop assembly (332) may stabilize cannula (130) during insertion. Additionally, suture loop assembly (332) maintains cannula (130) in a generally tangential orientation relative to the incision. Such tangential orientation may reduce trauma as cannula (130) is guided through the incision. As cannula (130) is inserted into the incision through suture loop assembly (332), an operator may use forceps or other instruments to further guide cannula (130) along an atraumatic path. Of course, use of forceps or other instruments is merely optional, and may be omitted in some examples. As noted above, a guide tack (or other device) may be used in lieu of suture loop assembly (332). Cannula (130) is advanced until distal end (132) is positioned near the targeted region of the subretinal space, on the opposite side of the choroid (306). Various suitable ways of visualizing distal end (132) to thereby observe proper positioning of distal end (132) will be apparent to those skilled in the art in view of the teachings herein.

Although not shown, in some examples, cannula (130) may include one or more markers on the surface of cannula (130) to indicate various depths of insertion. While merely optional, such markers may be desirable to aid an operator in identifying the proper depth of insertion as cannula (130) is guided along an atraumatic path. For instance, the operator may visually observe the position of such markers in relation to suture loop assembly (332) and/or in relation to the incision in the sclera (304) as an indication of the depth to which cannula (130) is inserted in eye (301). By way of example only, one such marker may correspond to an approximately 6 mm depth of insertion of cannula (130).

As shown in FIG. 7D, once cannula (130) is at least partially inserted into eye (301), an operator may insert an optical fiber (315) into eye chandelier port (314) if the fiber (315) had not yet been inserted at this stage. With eye chandelier port (314) in place and assembled with optical fiber (315), an operator may activate eye chandelier port (314) by directing light through optical fiber (315) to provide illumination of eye (301) and thereby visualize the interior of eye (301). Further adjustments to the positioning of cannula (130) may optionally be made at this point to ensure proper positioning relative to the area of geographic atrophy of retina (308). In some instances, the operator may wish to rotate the eye (301), such as by pulling on suture loop assembly (332), to direct the pupil of the eye (301) toward the operator in order to optimize visualization of the interior of the eye (301) via the pupil.

FIGS. 7C-7D show cannula (130) as it is guided between sclera (304) and choroid (306) to position distal end (132) of cannula (130) at the delivery site for the therapeutic agent. In the present example, the delivery site corresponds to a generally posterior region of eye (301) adjacent to an area of geographic atrophy of retina (308). In particular, the delivery site of the present example is superior to the macula, in the potential space between the neurosensory retina and the retinal pigment epithelium layer. By way of example only, the operator may rely on direct visualization through a microscope directed through the pupil of eye (301) as cannula (130) is being advanced through the range of motion shown in FIGS. 7C-7D, with illumination provided through fiber (315) and port (314). Cannula (130) may be at least partially visible through a retina (308) and choroid (306) of eye (301). Visual tracking may be enhanced in versions where an optical fiber is used to emit visible light through the distal end of cannula (130).

Once cannula (130) has been advanced to the delivery site as shown in FIG. 7D, an operator may advance needle (150) of instrument (100) as described above by actuating knob (120). As can be seen in FIG. 7E, needle (150) is advanced relative to cannula (130) such that needle (150) pierces through choroid (306) without penetrating retina (308). Immediately prior to penetrating choroid (306), needle (150) may appear under direct visualization as “tenting” the surface of choroid (306). In other words, needle (150) may deform choroid (306) by pushing upwardly on choroid (306), providing an appearance like a tent pole deforming the roof of a tent. Such a visual phenomenon may be used by an operator to identify whether choroid (306) is about to be pierced and the location of any eventual piercing. The particular amount of needle (150) advancement sufficient to initiate “tenting” and subsequent piercing of choroid (306) may be of any suitable amount as may be determined by a number of factors such as, but not limited to, general patient anatomy, local patient anatomy, operator preference, and/or other factors. As described above, an example of a range of needle (150) advancement may be between approximately 0.25 mm and approximately 10 mm; or more particularly between approximately 2 mm and approximately 6 mm.

In the present example, after the operator has confirmed that needle (150) has been properly advanced by visualizing the tenting effect described above, the operator infuses a balanced salt solution (BSS) or other similar solution as needle (150) is advanced relative to cannula (130). Such a BSS may form a leading bleb (340) ahead of needle (150) as needle (150) is advanced through choroid (306). Leading bleb (340) may be desirable for two reasons. First, as shown in FIG. 7F, leading bleb (340) may provide a further visual indicator to an operator to indicate when needle (150) is properly positioned at the delivery site. Second, leading bleb (340) may provide a barrier between needle (150) and retina (308) once needle (150) has penetrated choroid (306). Such a barrier may push the retinal wall outwardly, thereby minimizing the risk of retinal perforation as needle (150) is advanced to the delivery site. In some versions, a foot pedal is actuated in order to drive leading bleb (340) out from needle (150). Alternatively, other suitable features that may be used to drive leading bleb (340) out from needle (150) will be apparent to those skilled in the art in view of the teachings herein.

Once the operator visualizes leading bleb (340), the operator may cease infusion of BSS, leaving a pocket of fluid as can be seen in FIG. 7F. Next, a therapeutic agent (342) may be infused by actuating fluid delivery system (80) or some other fluid delivery device as described in various references cited herein. The delivered therapeutic agent (342) may be any suitable therapeutic agent configured to treat an ocular condition. Some merely illustrative examples of suitable therapeutic agents may include, but are not necessarily limited to, drugs having smaller or large molecules, therapeutic cell solutions, certain gene therapy solutions, tissue plasminogen activators, and/or any other suitable therapeutic agent as will be apparent to those skilled in the art in view of the teachings herein. By way of example only, the therapeutic agent (342) may be provided in accordance with at least some of the teachings of U.S. Pat. No. 7,413,734, entitled “Treatment of Retinitis Pigmentosa with Human Umbilical Cord Cells,” issued Aug. 19, 2008, the disclosure of which is incorporated by reference herein, in its entirety. In addition to, or as an alternative to, being used to deliver a therapeutic agent (342), instrument (100) and variations thereof may be used to provide drainage and/or perform other operations.

In the present example, the amount of therapeutic agent (342) that is ultimately delivered to the delivery site is approximately 50 μL, although any other suitable amount may be delivered. In some versions, a foot pedal is actuated in order to drive agent (342) out from needle (150). Alternatively, other suitable features that may be used to drive agent (342) out from needle (150) will be apparent to those skilled in the art in view of the teachings herein. Delivery of therapeutic agent (342) may be visualized by an expansion of the pocket of fluid as can be seen in FIG. 7G. As shown, therapeutic agent (342) essentially mixes with the fluid of leading bleb (340) as therapeutic agent (342) is injected into the subretinal space.

Once delivery is complete, needle (150) may be retracted by rotating knob (120) in a direction opposite to that used to advance needle (150); and cannula (130) may then be withdrawn from eye (301). Because of the size of needle (150), the site where needle (150) penetrated through choroid (306) is self-sealing, such that no further steps need be taken to seal the delivery site through choroid (306). Suture loop assembly (332) and chandelier (314) may be removed, and the incision in the sclera (304) may be closed using any suitable conventional techniques.

As noted above, the foregoing procedure may be carried out to treat a patient having macular degeneration. In some such instances, the therapeutic agent (342) that is delivered by needle (150) may comprise cells that are derived from postpartum umbilicus and placenta. As noted above, and by way of example only, the therapeutic agent (342) may be provided in accordance with at least some of the teachings of U.S. Pat. No. 7,413,734, the disclosure of which is incorporated by reference herein, in its entirety. Alternatively, needle (150) may be used to deliver any other suitable substance or substances, in addition to or in lieu of those described in U.S. Pat. No. 7,413,734 and/or elsewhere herein. By way of example only, therapeutic agent (342) may comprise various kinds of drugs including but not limited to small molecules, large molecules, cells, and/or gene therapies. It should also be understood that macular degeneration is just one merely illustrative example of a condition that may be treated through the procedure described herein. Other biological conditions that may be addressed using the instruments and procedures described herein will be apparent to those of ordinary skill in the art.

The procedure described above may be carried out in accordance with any of the teachings of U.S. Pat. No. 10,226,379, the disclosure of which is incorporated by reference herein, in its entirety; U.S. Pat. No. 9,949,874, entitled “Therapeutic Agent Delivery Device with Convergent Lumen,” issued Apr. 24, 2018, the disclosure of which is incorporated by reference herein, in its entirety; U.S. Pat. No. 9,925,088, entitled “Sub-Retinal Tangential Needle Catheter Guide and Introducer,” issued Mar. 27, 2018, the disclosure of which is incorporated by reference herein, in its entirety; U.S. Pat. No. 10,322,028, entitled “Method and Apparatus for Sensing Position Between Layers of an Eye,” issued Jun. 18, 2019, the disclosure of which is incorporated by reference herein, in its entirety; U.S. Pat. No. 10,064,752, entitled “Motorized Suprachoroidal Injection of Therapeutic Agent,” issued Sep. 4, 2018, the disclosure of which is incorporated by reference herein, in its entirety; U.S. Pat. No. 10,219,936, entitled “Therapeutic Agent Delivery Device with Advanceable Cannula and Needle,” issued Mar. 5, 2019, the disclosure of which is incorporated by reference herein, in its entirety; U.S. Pat. No. 10,258,502, entitled “Therapeutic Agent Delivery Device,” issued Apr. 16, 2019, the disclosure of which is incorporated by reference herein, in its entirety; and/or International Pub. No. WO 2022/136913, entitled “Ocular Cannula Guide,” published Jun. 30, 2022, the disclosure of which is incorporated by reference herein, in its entirety.

### III. EXAMPLE OF AN ALTERNATIVE CANNULA FOR INSTRUMENT

In some instances, it may be desirable to provide cannula (130) with an atraumatic wedge-shaped distal end, such as for assisting with insertion of cannula (130) through the sclerotomy incision and into the space between sclera (304) and choroid (306). It will be appreciated that such a wedge-shaped distal end may improve the ability of cannula (130) to provide separation between the sclera (304) and choroid (306) layers via blunt dissection and may thereby improve the ability of cannula (130) to be advanced between such layers while not inflicting trauma to the sclera or choroid layers.

In addition, or alternatively, it may be desirable to provide cannula (130) with a needle guide (also referred to as an insert) disposed within the needle guide lumen of cannula (130). In this regard, an example of a needle guide is described in U.S. Pat. No. 10,478,553, the disclosure of which is incorporated by reference herein, in its entirety. In some instances, it may be desirable for such a needle guide to be constructed of a material having a relatively low hardness, at least by comparison to the hardness of stainless steel, for example. It will be appreciated that such a relatively low hardness may improve the ability of cannula (130) to conform to the specific structures and contours of the eye (301) through lateral bending as cannula (130) is advanced toward the posterior region of the eye (301) between the sclera (304) and choroid (306) layers.

In addition, or alternatively, it may be desirable to provide cannula (130) with a varying stiffness along the length of cannula (130). For example, it may be desirable to provide a distal segment of cannula (130) near its distal end with a relatively high stiffness, such as to reduce any curvature that might otherwise be imparted to cannula (130) by needle (150) (e.g., in cases where needle (150) includes a preformed bend and/or curve), such as when needle (150) is retracted within cannula (130), and thereby promote the atraumatic passage of cannula (130) along the suprachoroidal space while needle (150) is retracted therein; and to provide a medial segment of cannula (130) that is proximal of the distal segment with a relatively low stiffness, such as to improve the ability of cannula (130) to conform to the specific structures and contours of the eye (301).

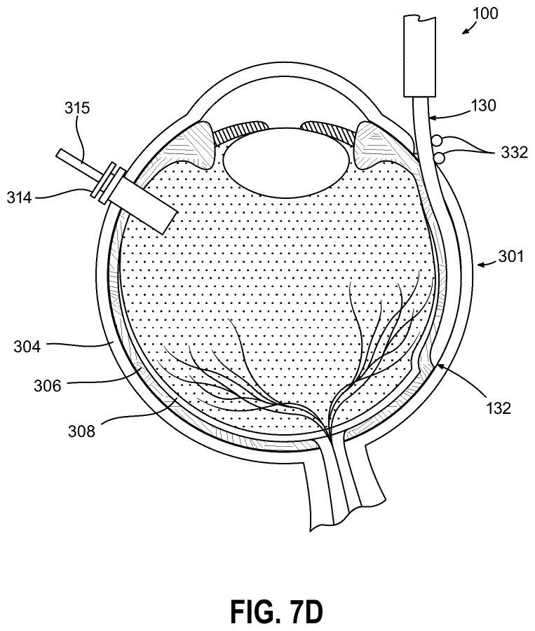

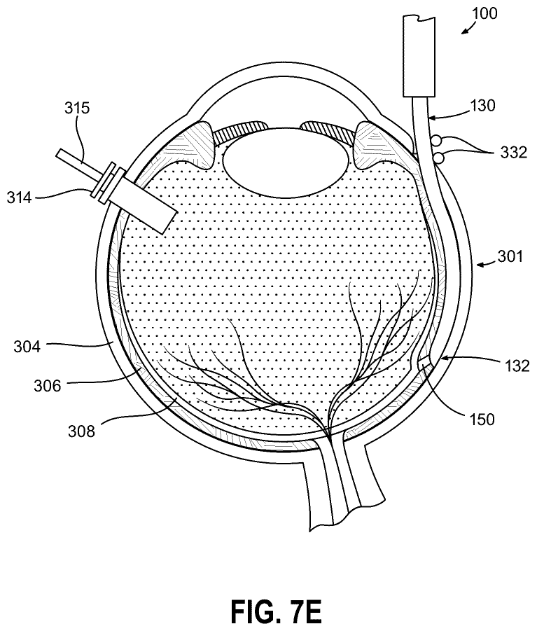

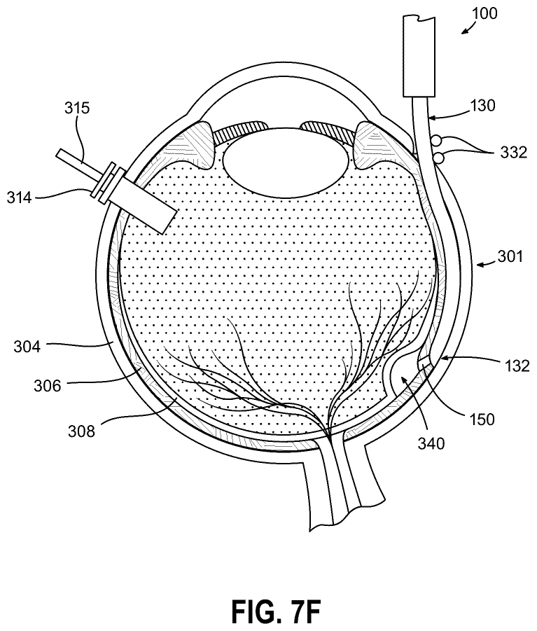

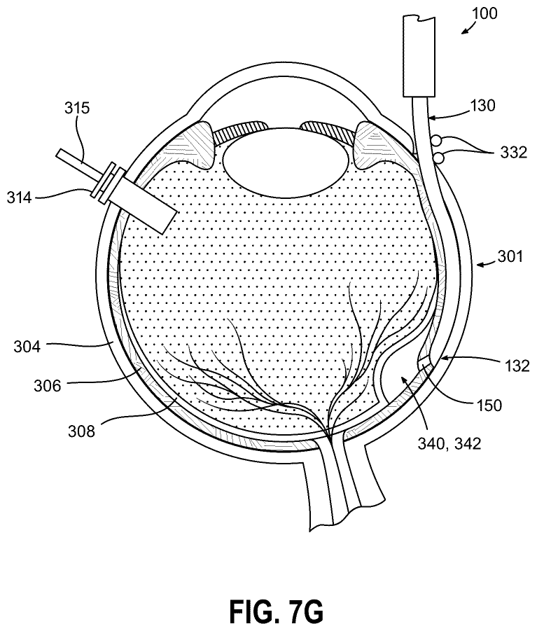

FIGS. 8-13 show an example of a cannula (430) that may function in this manner and that may be readily incorporated into instrument (100) in place of cannula (130). Cannula (430) may be similar to cannula (130) described above, except as otherwise described below. For instance, like with cannula (130), cannula (430) is flexible enough to conform to the specific structures and contours of the patient's eye; yet cannula (430) has sufficient column strength to permit advancement of cannula (430) between the sclera and choroid of the eye (301) without buckling. In some versions, cannula (430) comprises a flexible material having a greater hardness than that of the flexible material of cannula (130). For example, cannula (430) may comprise a flexible material having a greater hardness than that of Polyether block amide (PEBA), though any other suitable material or combination of materials may be used.

Cannula (430) of the present example includes a distal end (432) and a distally facing opening (434) near distal end (432). Opening (434) of the present example is adjacent to U-shaped lateral recess (436) in cannula (430), which leads to an open distal end (438) of a needle guide lumen (439) within cannula (430). Opening (434) is spaced proximally from the tip of distal end (432); while lateral recess (436) extends the length from opening (434) to the tip of distal end (432). In some versions, opening (434) is oriented along a plane that is perpendicular to the longitudinal axis of cannula (430). In some other versions, opening (434) is oriented along a plane that is obliquely oriented relative to the longitudinal axis of cannula (430). In either case, due to the position and configuration of opening (434) in combination with lateral recess (436), cannula (430) may be regarded as providing a path for transversely oriented exit of a needle (550, 650, 750, 850, 950, 1050, 1150) from cannula (430) as needle (550, 650, 750, 850, 950, 1050, 1150) is advanced distally from cannula (430) as described below.

Distal end (432) is atraumatic such that distal end (432) is configured to provide separation between the sclera (304) and choroid (306) layers via blunt dissection, to thereby enable cannula (430) to be advanced between such layers while not inflicting trauma to the sclera or choroid layers. In this regard, distal end (432) of the present example is defined by a longitudinally-extending lower surface (433) and an obliquely-extending upper surface (435) that tapers downwardly in the distal direction toward lower surface (433), such that distal end (432) is generally wedge-shaped. As shown, U-shaped lateral recess (436) extends through upper surface (435) of distal end (432) in the present example. It will be appreciated that distal end (432) may be provided with a wedge shape in any other suitable manner. As noted above, the wedge shaped of distal end (432) may improve the ability of cannula (430) to provide separation between the sclera (304) and choroid (306) layers via blunt dissection and may thereby improve the ability of cannula (430) to be advanced between such layers while not inflicting trauma to the sclera (304) and choroid (306) layers. The wedge shape of distal end (432) may also assist in maintaining the angular orientation of distal end (432) of cannula (430) about the longitudinal axis of cannula (430) as cannula (430) is advanced to toward the posterior region of the eye (301) between the sclera (304) and choroid (306) layers. In other words, wedge shape of distal end (432) may assist in maintaining the orientation of lateral recess (436) toward the interior region of the eye (301), to thereby promote the appropriate trajectory of needle (150, 550, 650, 750, 850, 950, 1050, 1150) toward the interior region of the eye (301) as needle (150, 550, 650, 750, 850, 950, 1050, 1150) is advanced distally from cannula (430).

In the example shown, cannula (430) has a varying cross-sectional area along a length of cannula (430), such that cannula (430) may likewise have a varying stiffness along the length of cannula (430). As best shown in FIGS. 8 and 10-11, cannula (430) of the present example includes a proximal segment (430p), a medial segment (430m), and a distal segment (430d). In the example shown, distal segment (430d) is immediately proximal of wedge-shaped distal end (432) of cannula (430), such that upper and lower surfaces of distal segment (430d) directly and continuously interface with upper and lower surfaces (433, 435) of distal end (432), respectively.

Proximal segment (430p) may have a generally rectangular (e.g., obround) cross-sectional profile and a first cross-sectional area, and may be configured to be manipulated by the operator for pushing and pulling medial and distal segments (430m, 430d) during use. While proximal segment (430p) of the present example has a generally rectangular cross-sectional profile, it will be appreciated that any other suitable cross-sectional profile (e.g., elliptical, etc.) may be used.

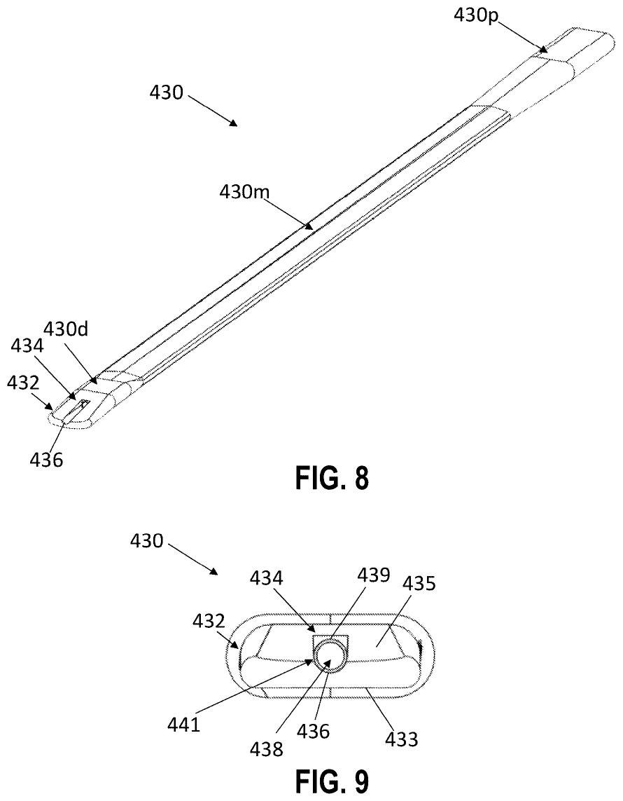

As shown in FIG. 12, medial segment (430m) has a generally diamond-shaped cross-sectional profile and a second cross-sectional area less than the first cross-sectional area. In this regard, cannula (430) may taper laterally and/transversely inwardly from proximal segment (430p) to medial segment (430m). While medial segment (430m) of the present example has a generally diamond-shaped cross-sectional profile, it will be appreciated that any other suitable cross-sectional profile (e.g., rectangular, elliptical, etc.) may be used. It will be appreciated that the decreased cross-sectional area of medial segment (430m) relative to proximal segment (430p) may provide medial segment (430m) with a lower stiffness than that of proximal segment (430p) to thereby contribute to the varying stiffness of cannula (430) along the length of cannula (430). As noted above, the relatively low stiffness of medial segment (430m) may improve the ability of cannula (430) to conform to the specific structures and contours of the eye (301). In some versions, medial segment (430m) has a length greater than the lengths of each of proximal and distal segments (430p, 430d). For example, medial segment (430m) may have a length greater than the combined lengths of proximal and distal segments (430p, 430d), and may comprise a majority of the overall length of cannula (430).

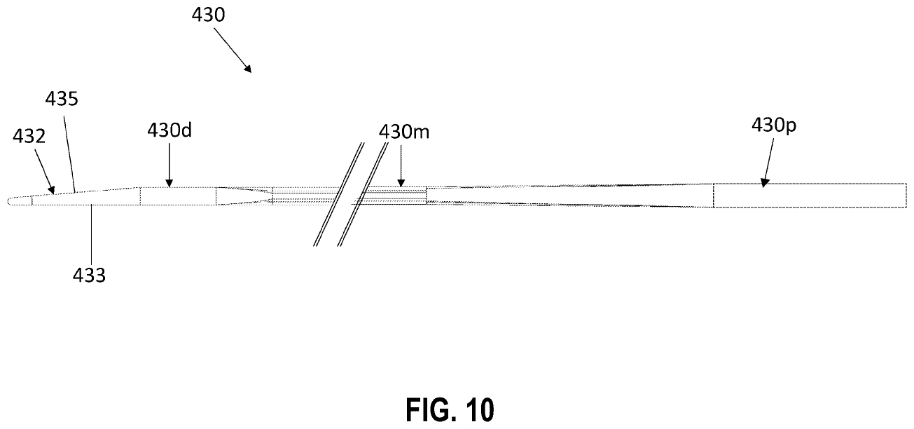

As shown in FIG. 13, distal segment (430d) has a generally rectangular (e.g., obround) cross-sectional profile and a third cross-sectional area greater than the second cross-sectional area. In this regard, cannula (430) may taper laterally and/or transversely outwardly from medial segment (430m) to distal segment (430d). In some versions, the third cross-sectional area may be less than the first cross-sectional area. While distal segment (430d) of the present example has a generally rectangular cross-sectional profile, it will be appreciated that any other suitable cross-sectional profile (e.g., elliptical, etc.) may be used.

It will be appreciated that the increased cross-sectional area of distal segment (430d) relative to medial segment (430m) may provide distal segment (430d) with a higher stiffness than that of medial segment (430m) to thereby contribute to the varying stiffness of cannula (430) along the length of cannula (430). In some versions, distal segment (430d) of cannula (430) may have a cross-sectional area substantially equal to that of cannula (130). For example, a width of distal segment (430d) may range from approximately 1.28 mm to approximately 1.92 mm; or may be more particularly approximately 1.6 mm; and/or a height of distal segment (430d) may range from approximately 0.48 mm to approximately 0.72 mm; or may be more particularly approximately 0.6 mm. As noted above, cannula (430) may also comprise a flexible material having a greater hardness than that of the material of cannula (130). Thus, distal segment (430d) of cannula (430) may have an increased stiffness relative to that of cannula (130). For example, distal segment (430d) may have a higher stiffness than that of cannula (130) at or near distal end (132) of cannula (130). As noted above, the relatively high stiffness of distal segment (430d) may reduce any curvature that might be imparted to cannula (430) by a needle retracted within cannula (430), such as needle (150), and thereby promote the atraumatic passage of cannula (430) along the suprachoroidal space while needle (150) is retracted therein.

In the present example, the generally rectangular, generally elliptical, or otherwise generally flat cross-sectional profile of distal segment (430d) of cannula (430) prevents cannula (430) from rotating about the longitudinal axis of cannula (430) when cannula (430) is disposed in the suprachoroidal space.

In other words, cross-sectional profile of distal segment (430d) of cannula (430) may assist in maintaining the orientation of lateral recess (436) toward the interior region of the eye (301), to thereby promote the appropriate trajectory of needle (550, 650, 750, 850, 950, 1050, 1150) toward the interior region of the eye (301) as needle (550, 650, 750, 850, 950, 1050, 1150) is advanced distally from cannula (430). Thus, the combination of the wedge shape of distal end (432) and the cross-sectional profile of distal segment (430d) may provide a consistent and predictable exit path for a needle (150, 550, 650, 750, 850, 950, 1050, 1150) when needle (150, 550, 650, 750, 850, 950, 1050, 1150) is advanced distally relative to cannula (430). The cross-sectional profiles of proximal segment (430p) and/or medial segment (430m) may provide similar effects.

In the example shown, a needle guide (441) is disposed within needle guide lumen (439) of cannula (430). Needle guide (441) may be secured within needle guide lumen (439) of cannula (430) by a press or interference fit, by adhesives, by mechanical locking mechanisms, and/or in any other suitable fashion. In the present example, needle guide (441) is formed of a polyimide material, though it should be understood that any other suitable biocompatible material(s) may be used, such as any other suitable biocompatible material(s) having a hardness less than that of stainless steel. Needle guide (441) of the present example is substantially straight yet may bend with cannula (430). Needle guide (441) defines a needle lumen (443) configured to slidably receive a needle, such as any of the needles (150, 550, 650, 750, 850, 950, 1050, 1150) described herein. As noted above, the relatively low hardness of the material of needle guide (441) may improve the ability of cannula (430) to conform to the specific structures and contours of the eye (301).

### IV. EXAMPLES OF ALTERNATIVE NEEDLES FOR INSTRUMENT

In some instances, it may be desirable to provide needle (150) with one or more preformed curved portions such that needle (150) may impart at least some degree of curvature to cannula (130) when needle (150) is slidably disposed therein. Such curved portions may improve the ability of needle (150) to access the subretinal space of an eye (301) that is relatively small (e.g., about 16 mm in diameter, or otherwise less than about 24 mm in diameter); at least by comparison to the eye (301) of an adult human patient (e.g., an eye of a pediatric human patient). Such curved portions of a needle (150) may improve the ability of needle (150) and/or cannula (130) to conform to the specific structures and contours of the eye (301). In addition, or alternatively, such curved portions may inhibit inadvertent movement of distal tip (152) of needle (150) that might otherwise result from movement of body (110) of instrument (100). It will be appreciated that by inhibiting such inadvertent movement of distal tip (152), such curved portions may assist with consistently maintaining distal tip (152) along a predetermined trajectory and angled at a predetermined orientation while within the eye (301), thereby improving the ability of needle (150) to access the subretinal space of the eye (301).

Various illustrative examples of such needles (550, 650, 750, 850, 950, 1050, 1150) are described in greater detail below. While needles (550, 650, 750, 850, 950, 1050, 1150) are described below in connection with cannula (130), it will be appreciated that any of needles (550, 650, 750, 850, 950, 1050, 1150) may be used with cannula (430). For example, any of needles (550, 650, 750, 850, 950, 1050, 1150) may be readily incorporated into instrument (100) in place of needle (150), and cannula (430) may be readily incorporated into instrument (100) in place of cannula (130). While the example described above is provided in the context of a relatively small eye (301), the teachings below may also be employed in the context of an eye (301) of an adult human patient, such that the teachings below are not limited to the context of a relatively small eye (301).

A. First Example of a Needle with Proximal Curved Portion and Distal Curved Portion

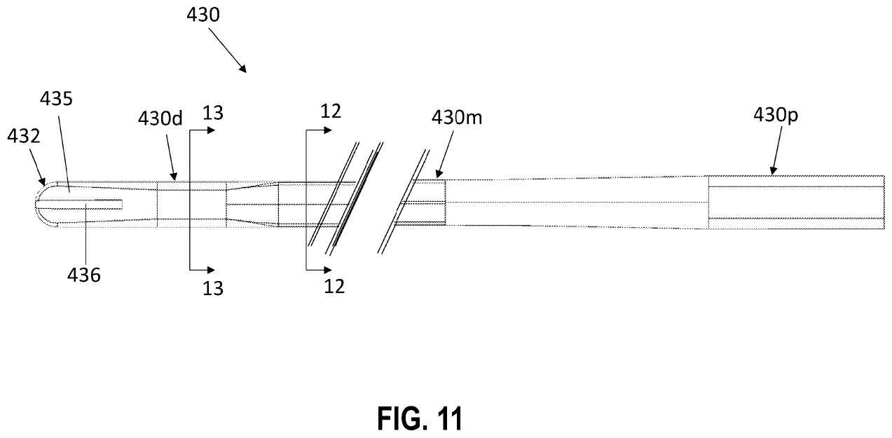

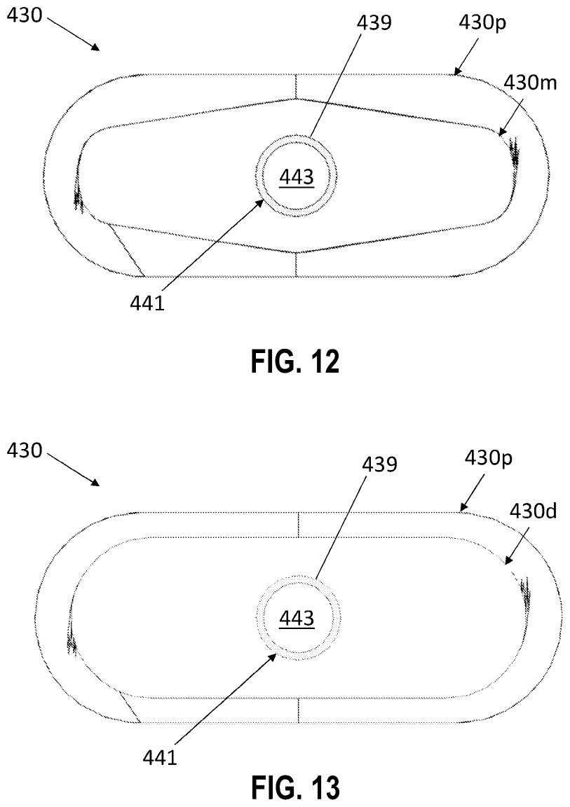

FIGS. 14-15 show an example of a needle (550) that may be readily incorporated into instrument (100) in place of needle (150). Needle (550) may be similar to needle (150) described above, except as otherwise described below. In this regard, needle (550) of the present example has a sharp distal tip (552) and defines a lumen (not shown). Distal tip (552) of the present example has a single bevel configuration. In some other versions, distal tip (552) has a tri-bevel configuration or any other suitable configuration such as any of those described in U.S. Pat. No. 10,226,379, the disclosure of which is incorporated by reference herein, in its entirety. Still other suitable forms that distal tip (552) may take will be apparent to those skilled in the art in view of the teachings herein. In the present example, needle (550) is formed of nitinol, though it should be understood that any other suitable material(s) (e.g., stainless steel, etc.) may be used.

Needle (550) of the present example includes a substantially straight proximal portion (560), a substantially curved proximal portion (562), a substantially straight medial portion (564), a substantially curved distal portion (566), and a substantially straight distal portion (568). Proximal curved portion (562) is longitudinally interposed between proximal straight portion (560) and medial straight portion (564); medial straight portion (564) is longitudinally interposed between proximal curved portion (562) and distal curved portion (566); distal curved portion (566) is longitudinally interposed between medial straight portion (564) and distal straight portion (568); and distal straight portion (568) is longitudinally interposed between distal curved portion (566) and distal tip (552). In the example shown, proximal curved portion (562) and distal curved portion (566) are curved in opposite directions from each other to provide needle (550) with a generally S-shaped configuration. For example, proximal curved portion (562) curves generally clockwise in the distal direction while distal curved portion (566) curves generally counterclockwise in the distal direction, within the frame of reference of FIGS. 14-15.

Proximal straight portion (560) may be housed within body (110) of instrument (100) to facilitate actuation of needle (550) via actuation knob (120), for example, and thus may be configured to remain external to the eye (301) during use. In some other versions, at least part of proximal straight portion (560) may be housed within a proximal portion of cannula (130, 430) yet may still remain external to the eye (301) during use. Proximal curved portion (562) may likewise be configured to remain external to the eye (301) during use, while each of medial straight portion (564), distal curved portion (566), and distal straight portion (568) may be configured to be at least partially positioned within the eye (301) during use. For example, at least medial straight portion (564) may be configured to conform to a curvature of the eye (301), together with the portion of cannula (130, 430) in which medial straight portion (564) is disposed. In addition, or alternatively, proximal curved portion (562) may be configured to remain disposed within cannula (130, 430) when needle (550) is advanced distally relative to cannula (130, 430) such as during initial tenting and/or subsequent piercing of choroid (306).

It should be understood that a portion of needle (550) may be “positioned within the eye (301) during use” even if that portion of needle (550) is still within cannula (130, 430), provided that the corresponding portion of cannula (130, 430) is positioned within the eye (301). Thus, a portion of needle (550) need not necessarily be distally exposed relative to cannula (130, 430) in order for that portion of needle (550) to be “positioned within the eye (301) during use.”

Needle (550) is configured to provide proximal curved portion (562) and distal curved portion (566) as preformed features, such that needle (550) is resiliently biased to assume the generally S-shaped configuration shown in FIG. 14. In the example shown, proximal straight portion (560) has a length (L1); proximal curved portion (562) has a constant radius of curvature (R1), an arclength (S1), and a center of curvature (C1) that is located at a distance (X1) from a proximal end of needle (550) along a first (e.g., horizontal) axis and at a distance (Y1) from the proximal end of needle (550) along a second (e.g., vertical) axis; medial straight portion (564) has a length (L2); distal curved portion (566) has a constant radius of curvature (R2) and an arclength (S2); and distal straight portion (568) has a length (L3).

By way of example only, length (L1) may range from approximately 11.52 mm to approximately 17.28 mm, or may be more particularly approximately 14.4 mm; radius of curvature (R1) may range from approximately 14.4 mm to approximately 21.6 mm, or may be more particularly approximately 18 mm; arclength (S1) may range from approximately 16 mm to approximately 24 mm, or may be more particularly approximately 20 mm; distance (X1) may range from approximately 11.52 mm to approximately 17.28 mm, or may be more particularly approximately 14.4 mm; distance (Y1) may range from approximately 14.36 mm to approximately 21.54 mm, or may be more particularly approximately 17.95 mm; length (L2) may range from approximately 32 mm to approximately 48 mm, or may be more particularly approximately 40 mm; radius of curvature (R2) may range from approximately 2.8 mm to approximately 4.2 mm, or may be more particularly approximately 3.5 mm; arclength (S2) may range from approximately 3.36 mm to approximately 5.04 mm, or may be more particularly approximately 4.2 mm; and/or length (L3) may range from approximately 0.56 mm to approximately 0.84 mm, or may be more particularly approximately 0.7 mm.

Proximal straight portion (560) may be configured to extend proximally away from the eye (301) and toward body (110) of instrument (100) while medial straight portion (564), distal curved portion (566), and/or distal straight portion (568) are disposed within the eye (301). In this regard, while medial straight portion (564) and the portion of cannula (130, 430) in which medial straight portion (564) is disposed may conform to a curvature of the eye (301), proximal curved portion (562) may be curved in the opposite direction and may impart at least some degree of curvature to cannula (130, 430), such that the portion of cannula (130, 430) external to the eye (301) may likewise be curved in the opposite direction. For example, at any one or more of the stages shown in FIGS. 7C-7G, proximal curved portion (562) may cause the portion of cannula (130, 430) external to the eye (301) to curve generally clockwise in the proximal direction away from the eye (301) and toward body (110), within the frame of reference of FIGS. 7C-7G.

Such induced curvature of cannula (130, 430) by proximal curved portion (562) may limit the impact of any movement of body (110) of instrument (100) on the position of distal tip (552); and may thereby inhibit inadvertent movement of distal tip (552) that might otherwise result from movement of body (110). For example, proximal curved portion (562) and/or the portion of cannula (130, 430) in which proximal curved portion (562) is disposed may effectively absorb some or all of such movement instead of transmitting such movement distally to medial straight portion (564), distal curved portion (566), and distal straight portion (568) of needle (550). In addition, or alternatively, the induced curvature of cannula (130, 430) by proximal curved portion (562) may further assist in the portion of cannula (130, 430) within the eye (301) conforming to the curvature of the eye (301).

While needle (550) of the present example includes a single proximal curved portion (562) having a constant radius of curvature (R1), in some other versions the radius of curvature (R1) may be variable and/or more than one proximal curved portion (562) may be provided, such as described in greater detail below.

Distal curved portion (566) may be configured to orient distal tip (552) along one or more predetermined exit axes during distal advancement of needle (550) relative to cannula (130, 430) to protrude from opening (134). For example, distal curved portion (566) may be configured and operable in accordance with at least some of the teachings of U.S. Pat. No. 10,478,553, the disclosure of which is incorporated by reference herein, in its entirety.

B. Second Example of a Needle with Proximal Curved Portion and Distal Curved Portion

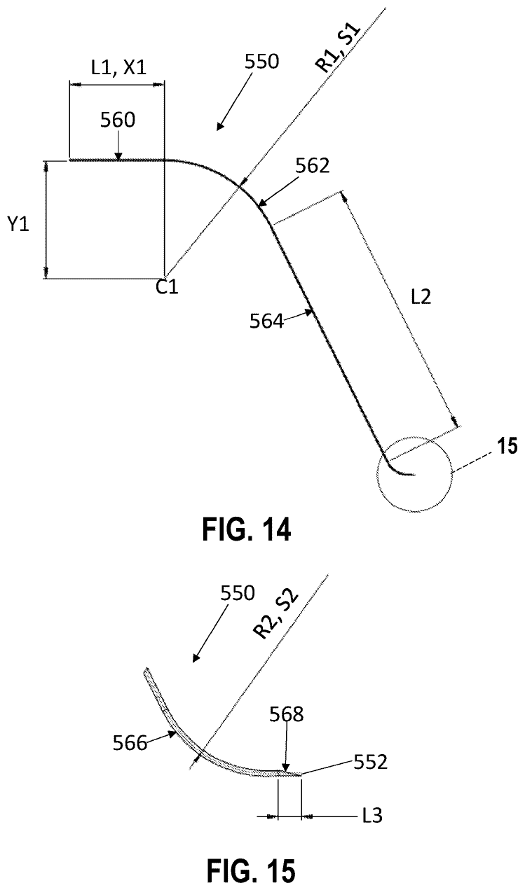

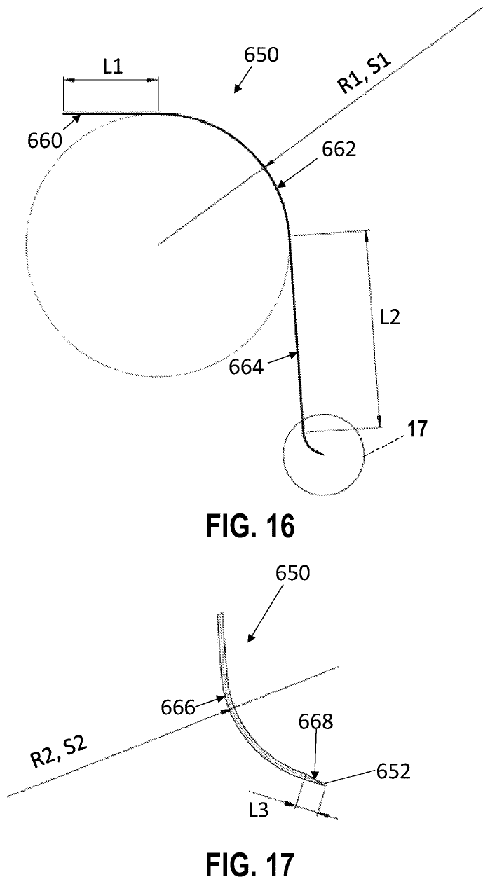

FIGS. 16-17 show another example of a needle (650) that may be readily incorporated into instrument (100) in place of needle (150). Needle (650) may be similar to needle (550) described above, except as otherwise described below. In this regard, needle (650) of the present example has a sharp distal tip (652) and defines a lumen (not shown).

Needle (650) of the present example includes a substantially straight proximal portion (660), a substantially curved proximal portion (662), a substantially straight medial portion (664), a substantially curved distal portion (666), and a substantially straight distal portion (668). In the example shown, proximal curved portion (662) and distal curved portion (666) are curved in opposite directions from each other to provide needle (650) with a generally S-shaped configuration. For example, proximal curved portion (662) curves generally clockwise in the distal direction while distal curved portion (666) curves generally counterclockwise in the distal direction, within the frame of reference of FIGS. 16-17.

Needle (650) is configured to provide proximal curved portion (662) and distal curved portion (666) as preformed features, such that needle (650) is resiliently biased to assume the generally S-shaped configuration shown in FIG. 16. In the example shown, proximal straight portion (660) has a length (L1); proximal curved portion (662) has a constant radius of curvature (R1) and an arclength (S1), medial straight portion (664) has a length (L2); distal curved portion (666) has a constant radius of curvature (R2) and an arclength (S2); and distal straight portion (668) has a length (L3).

By way of example only, length (L1) may range from approximately 11.52 mm to approximately 17.28 mm, or may be more particularly approximately 14.4 mm; radius of curvature (R1) may range from approximately 16 mm to approximately 24 mm, or may be more particularly approximately 20 mm; arclength (S1) may range from approximately 24 mm to approximately 36 mm, or may be more particularly approximately 30 mm; length (L2) may range from approximately 24 mm to approximately 36 mm, or may be more particularly approximately 30 mm; radius of curvature (R2) may range from approximately 2.8 mm to approximately 4.2 mm, or may be more particularly approximately 3.5 mm; arclength (S2) may range from approximately 3.36 mm to approximately 5.04 mm, or may be more particularly approximately 4.2 mm; and/or length (L2) may range from approximately 0.56 mm to approximately 0.84 mm, or may be more particularly approximately 0.7 mm.

In some versions, proximal curved portion (662) may cause the portion of cannula (130, 430) external to the eye (301) to curve away from the eye (301) and toward body (110), in a manner similar to that described above in connection with FIG. 14. Such induced curvature of cannula (130, 430) by proximal curved portion (662) may limit the impact of any movement of body (110) of instrument (100) on the position of distal tip (652); and may thereby inhibit inadvertent movement of distal tip (652) that might otherwise result from movement of body (110). For example, proximal curved portion (662) and/or the portion of cannula (130, 430) in which proximal curved portion (662) is disposed may effectively absorb some or all of such movement instead of transmitting such movement distally to medial straight portion (664), distal curved portion (666), and distal straight portion (668) of needle (650). In addition, or alternatively, the induced curvature of cannula (130, 430) by proximal curved portion (662) may further assist in the portion of cannula (130, 430) within the eye (301) conforming to the curvature of the eye (301).

C. Third Example of a Needle with Proximal Curved Portion and Distal Curved Portion

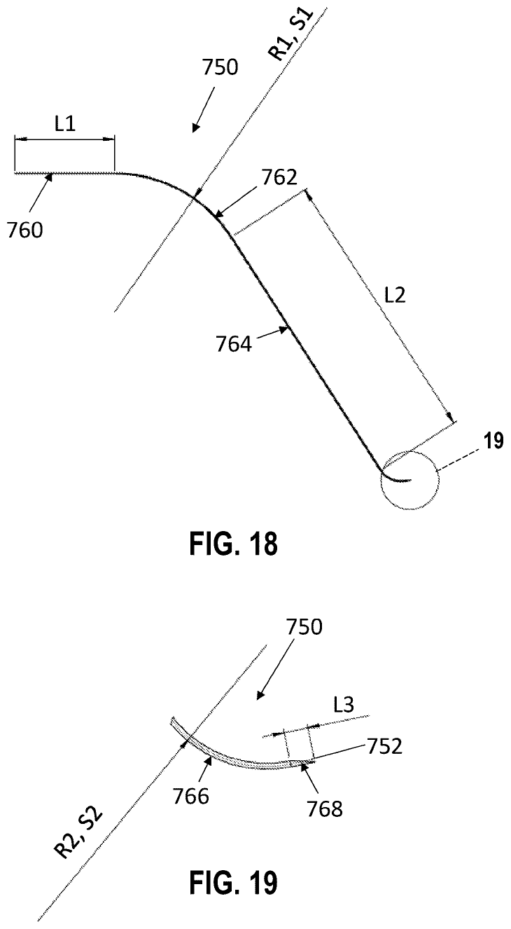

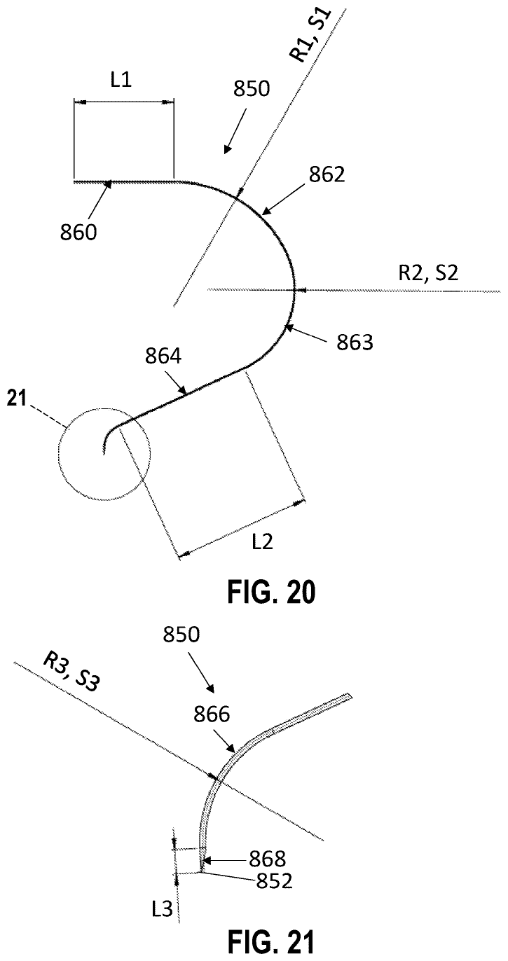

FIGS. 18-19 show another example of a needle (750) that may be readily incorporated into instrument (100) in place of needle (150). Needle (750) may be similar to needle (550) described above, except as otherwise described below. In this regard, needle (750) of the present example has a sharp distal tip (752) and defines a lumen (not shown).

Needle (750) of the present example includes a substantially straight proximal portion (760), a substantially curved proximal portion (762), a substantially straight medial portion (764), a substantially curved distal portion (766), and a substantially straight distal portion (768). In the example shown, proximal curved portion (762) and distal curved portion (766) are curved in opposite directions from each other to provide needle (750) with a generally S-shaped configuration. For example, proximal curved portion (762) curves generally clockwise in the distal direction while distal curved portion (766) curves generally counterclockwise in the distal direction, within the frame of reference of FIGS. 18-19.

Needle (750) is configured to provide proximal curved portion (762) and distal curved portion (766) as preformed features, such that needle (750) is resiliently biased to assume the generally S-shaped configuration shown in FIG. 18. In the example shown, proximal straight portion (760) has a length (L1); proximal curved portion (762) has a constant radius of curvature (R1) and an arclength (S1), medial straight portion (764) has a length (L2); distal curved portion (766) has a constant radius of curvature (R2) and an arclength (S2); and distal straight portion (768) has a length (L3).

By way of example only, length (L1) may range from approximately 11.52 mm to approximately 17.28 mm, or may be more particularly approximately 14.4 mm; radius of curvature (R1) may range from approximately 16 mm to approximately 24 mm, or may be more particularly approximately 20 mm; arclength (S1) may range from approximately 16 mm to approximately 24 mm, or may be more particularly approximately 20 mm; length (L2) may range from approximately 32 mm to approximately 48 mm, or may be more particularly approximately 40 mm; radius of curvature (R2) may range from approximately 2.8 mm to approximately 4.2 mm, or may be more particularly approximately 3.5 mm; arclength (S2) may range from approximately 3.36 mm to approximately 5.04 mm, or may be more particularly approximately 4.2 mm; and/or length (L2) may range from approximately 0.56 mm to approximately 0.84 mm, or may be more particularly approximately 0.7 mm.

In some versions, proximal curved portion (762) may cause the portion of cannula (130, 430) external to the eye (301) to curve away from the eye (301) and toward body (110), in a manner similar to that described above in connection with FIG. 14. Such induced curvature of cannula (130, 430) by proximal curved portion (762) may limit the impact of any movement of body (110) of instrument (100) on the position of distal tip (752); and may thereby inhibit inadvertent movement of distal tip (752) that might otherwise result from movement of body (110). For example, proximal curved portion (762) and/or the portion of cannula (130, 430) in which proximal curved portion (762) is disposed may effectively absorb some or all of such movement instead of transmitting such movement distally to medial straight portion (764), distal curved portion (766), and distal straight portion (768) of needle (750). In addition, or alternatively, the induced curvature of cannula (130, 430) by proximal curved portion (762) may further assist in the portion of cannula (130, 430) within the eye (301) conforming to the curvature of the eye (301).

D. Example of a Needle with First and Second Proximal Curved Portions and Distal Curved Portion

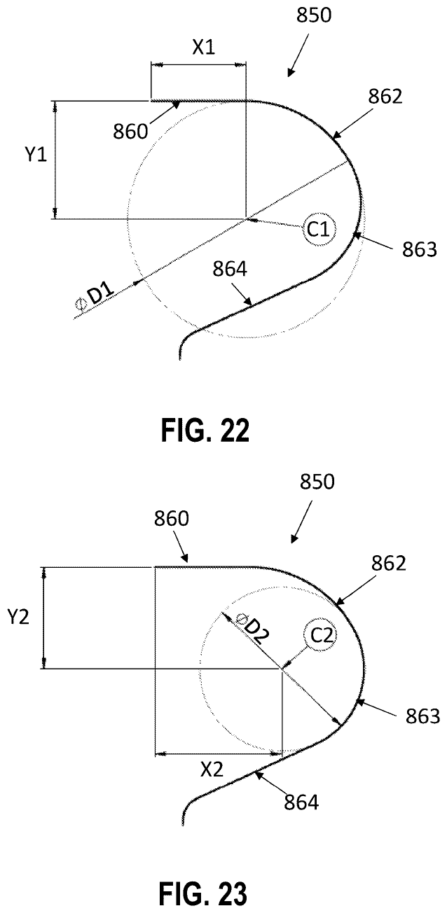

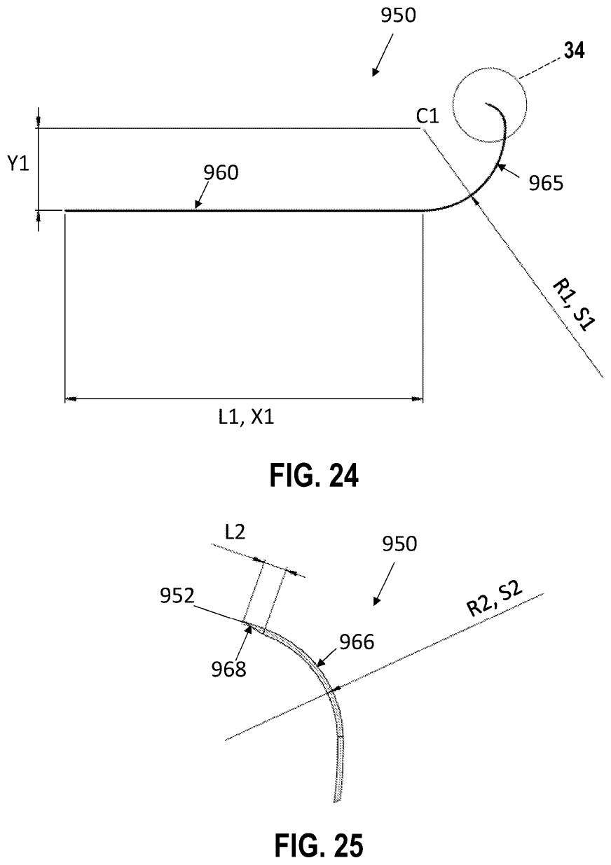

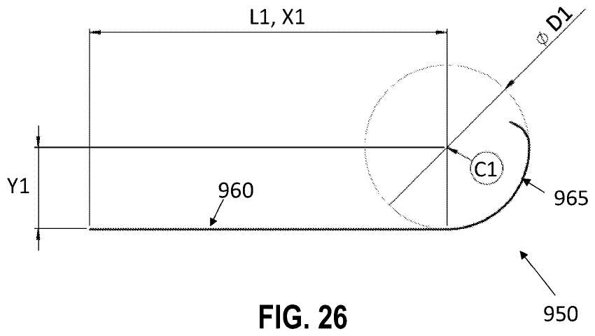

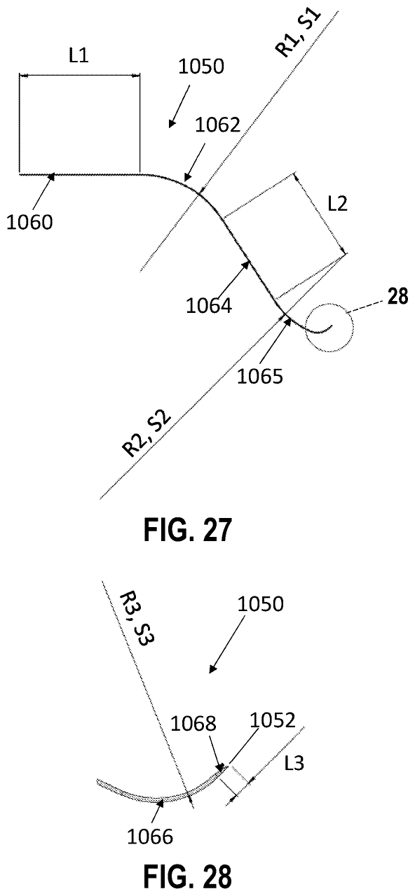

FIGS. 20-23 show another example of a needle (850) that may be readily incorporated into instrument (100) in place of needle (150). Needle (850) may be similar to needle (550) described above, except as otherwise described below. In this regard, needle (850) of the present example has a sharp distal tip (852) and defines a lumen (not shown).

Needle (850) of the present example includes a substantially straight proximal portion (860), a first substantially curved proximal portion (862), a second substantially curved proximal portion (863), a substantially straight medial portion (864), a substantially curved distal portion (866), and a substantially straight distal portion (868). First proximal curved portion (862) is longitudinally interposed between proximal straight portion (860) and second proximal curved portion (863); second proximal curved portion (863) is longitudinally interposed between first proximal curved portion (862) and medial straight portion (864); medial straight portion (864) is longitudinally interposed between second proximal curved portion (863) and distal curved portion (866); distal curved portion (866) is longitudinally interposed between medial straight portion (864) and distal straight portion (868); and distal straight portion (868) is longitudinally interposed between distal curved portion (866) and distal tip (852). In the example shown, proximal curved portions (862, 863) and distal curved portion (866) are curved in opposite directions from each other to provide needle (850) with a generally S-shaped configuration. For example, proximal curved portions (862, 863) each curve generally clockwise in the distal direction while distal curved portion (866) curves generally counterclockwise in the distal direction, within the frame of reference of FIGS. 20-23.

Needle (850) is configured to provide proximal curved portions (862, 863) and distal curved portion (866) as preformed features, such that needle (850) is resiliently biased to assume the generally S-shaped configuration shown in FIGS. 20 and 22-23. In the example shown, proximal straight portion (860) has a length (L1); first proximal curved portion (862) has a constant radius of curvature (R1), an arclength (S1), and a center of curvature (C1) that is located at a distance (X1) from a proximal end of needle (850) along a first (e.g., horizontal) axis and at a distance (Y1) from the proximal end of needle (850) along a second (e.g., vertical) axis, and is defined by a reference circle having center (C1) and a first diameter (D1); second proximal curved portion (863) has a constant radius of curvature (R2), an arclength (S2), and a center of curvature (C2) that is located at a distance (X2) from a proximal end of needle (850) along a first (e.g., horizontal) axis and at a distance (Y2) from the proximal end of needle (850) along a second (e.g., vertical) axis, and is defined by a reference circle having center (C2) and a second diameter (D2); medial straight portion (864) has a length (L2); distal curved portion (866) has a constant radius of curvature (R3) and an arclength (S3); and distal straight portion (868) has a length (L3).

By way of example only, length (L1) may range from approximately 11.52 mm to approximately 17.28 mm, or may be more particularly approximately 14.4 mm; radius of curvature (R1) may range from approximately 14.4 mm to approximately 21.6 mm, or may be more particularly approximately 18 mm; arclength (S1) may range from approximately 16 mm to approximately 24 mm, or may be more particularly approximately 20 mm; distance (X1) may range from approximately 11.52 mm to approximately 17.28 mm, or may be more particularly approximately 14.4 mm; distance (Y1) may range from approximately 14.4 mm to approximately 21.6 mm, or may be more particularly approximately 18 mm; diameter (D1) may range from approximately 28.8 mm to approximately 43.2, or may be more particularly approximately 36 mm; radius of curvature (R2) may range from approximately 10 mm to approximately 15 mm, or may be more particularly approximately 12.5 mm; arclength (S2) may range from approximately 16 mm to approximately 24 mm, or may be more particularly approximately 20 mm; distance (X2) may range from approximately 15.46 mm to approximately 23.2 mm, or may be more particularly approximately 19.33 mm; distance (Y1) may range from approximately 12.45 mm to approximately 18.67 mm, or may be more particularly approximately 15.56 mm; diameter (D2) may range from approximately 20 mm to approximately 30 mm, or may be more particularly approximately 25 mm; length (L2) may range from approximately 16 mm to approximately 24 mm, or may be more particularly approximately 20 mm; radius of curvature (R3) may range from approximately 2.8 mm to approximately 4.2 mm, or may be more particularly approximately 3.5 mm; arclength (S3) may range from approximately 3.36 mm to approximately 5.04 mm, or may be more particularly approximately 4.2 mm; and/or length (L3) may range from approximately 0.56 mm to approximately 0.84 mm, or may be more particularly approximately 0.7 mm.

In some versions, one or both proximal curved portions (862, 863) may cause the portion of cannula (130, 430) external to the eye (301) to curve away from the eye (301) and toward body (110), in a manner similar to that described above in connection with FIG. 14. Such induced curvature of cannula (130, 430) by one or both proximal curved portions (862, 863) may limit the impact of any movement of body (110) of instrument (100) on the position of distal tip (852); and may thereby inhibit inadvertent movement of distal tip (852) that might otherwise result from movement of body (110). For example, one or both proximal curved portions (862, 863) and/or the portion of cannula (130, 430) in which one or both proximal curved portions (862, 863) is disposed may effectively absorb some or all of such movement instead of transmitting such movement distally to medial straight portion (864), distal curved portion (866), and distal straight portion (868) of needle (850).

In addition, or alternatively, the induced curvature of cannula (130, 430) by one or both proximal curved portions (862, 863) may further assist in the portion of cannula (130, 430) within the eye (301) conforming to the curvature of the eye (301).

E. Example of a Needle with First and Second Distal Curved Portions

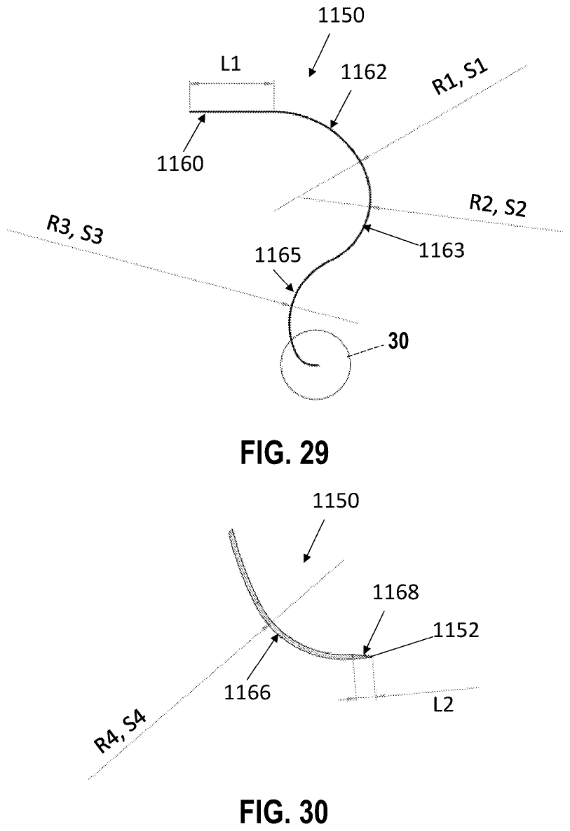

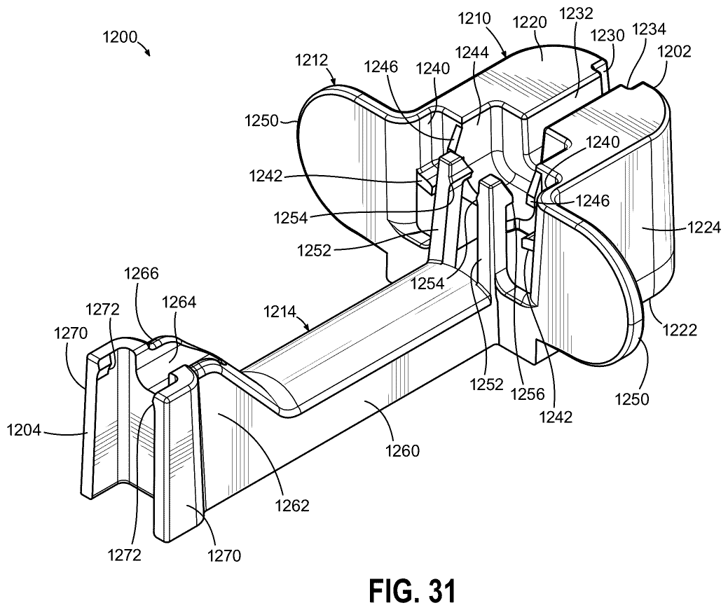

FIGS. 24-26 show another example of a needle (950) that may be readily incorporated into instrument (100) in place of needle (150). Needle (950) may be similar to needle (150) described above, except as otherwise described below. In this regard, needle (950) of the present example has a sharp distal tip (952) and defines a lumen (not shown). Distal tip (952) of the present example has a single bevel configuration. In some other versions, distal tip (952) has a tri-bevel configuration or any other suitable configuration such as any of those described in U.S. Pat. No. 10,226,379, the disclosure of which is incorporated by reference herein, in its entirety. Still other suitable forms that distal tip (952) may take will be apparent to those skilled in the art in view of the teachings herein. In the present example, needle (950) is formed of nitinol, though it should be understood that any other suitable material(s) (e.g., stainless steel, etc.) may be used.

Needle (950) of the present example includes a substantially straight proximal portion (960), a first substantially curved distal portion (965), a second substantially curved distal portion (966), and a substantially straight distal portion (968). First distal curved portion (965) is longitudinally interposed between proximal straight portion (960) and second distal curved portion (966); second distal curved portion (966) is longitudinally interposed between first distal curved portion (965) and distal straight portion (968); and distal straight portion (968) is longitudinally interposed between second distal curved portion (966) and distal tip (952). In the example shown, distal curved portions (965, 966) are curved in a same direction as each other to provide needle (950) with a generally J-shaped configuration. For example, distal curved portions (965, 966) each curve generally counterclockwise in the distal direction, within the frame of reference of FIGS. 24-26.

Each of proximal straight portion (960), distal curved portions (965, 966), and distal straight portion (968) may be configured to be at least partially positioned within the eye (301) during use. For example, at least proximal straight portion (960) may be configured to conform to a curvature of the eye (301), together with the portion of cannula (130, 430) in which proximal straight portion (960) is disposed. In addition, or alternatively, first distal curved portion (965) may have a radius of curvature that is substantially equal to that of the eye (301).

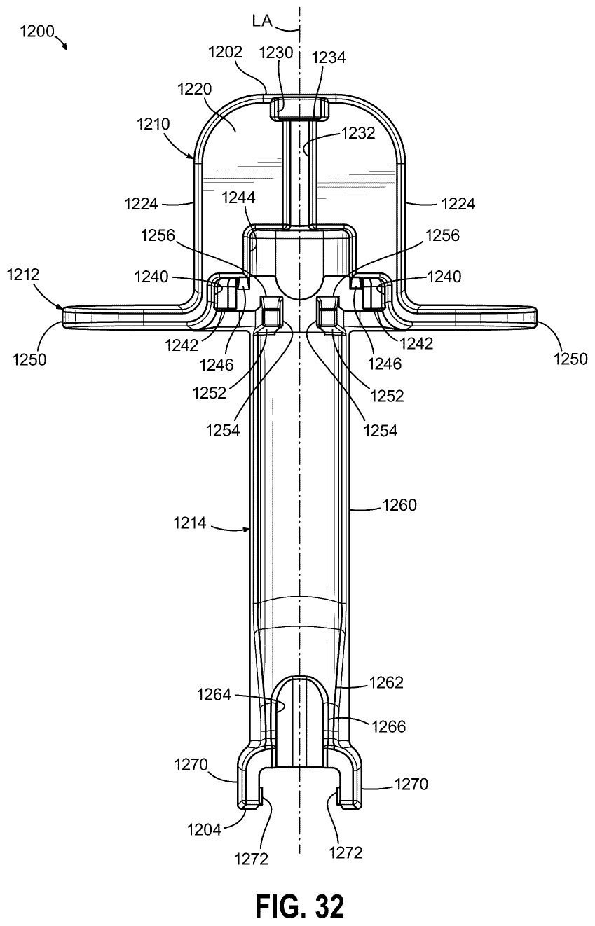

Needle (950) is configured to provide distal curved portions (965, 966) as preformed features, such that needle (950) is resiliently biased to assume the generally J-shaped configuration shown in FIGS. 24 and 26. In the example shown, proximal straight portion (960) has a length (L1); first distal curved portion (965) has a constant radius of curvature (R1), an arclength (S1), and a center of curvature (C1) that is located at a distance (X1) from a proximal end of needle (950) along a first (e.g., horizontal) axis and at a distance (Y1) from the proximal end of needle (950) along a second (e.g., vertical) axis, and is defined by a reference circle having center (C1) and a diameter (D1); second distal curved portion (966) has a constant radius of curvature (R2) and an arclength (S2); and distal straight portion (968) has a length (L2).

By way of example only, length (L1) may range from approximately 43.52 mm to approximately 65.28 mm, or may be more particularly approximately 54.4 mm; radius of curvature (R1) may range from approximately 10 mm to approximately 15 mm, or may be more particularly approximately 12.5 mm; arclength (S1) may range from approximately 16 mm to approximately 24 mm, or may be more particularly approximately 20 mm; distance (X1) may range from approximately 43.52 mm to approximately 65.28 mm, or may be more particularly approximately 54.4 mm; distance (Y1) may range from approximately 9.94 mm to approximately 14.9 mm, or may be more particularly approximately 12.42 mm; D1 may range from approximately 20 mm to approximately 30 mm, or may be more particularly approximately 25 mm; radius of curvature (R2) may range from approximately 2.8 mm to approximately 4.2 mm, or may be more particularly approximately 3.5 mm; arclength (S2) may range from approximately 3.36 mm to approximately 5.04 mm, or may be more particularly approximately 4.2 mm; and/or length (L2) may range from approximately 0.56 mm to approximately 0.84 mm, or may be more particularly approximately 0.7 mm.

In some versions, first distal curved portion (965) may impart at least some degree of curvature to cannula (130, 430), such as at any one or more of the stages shown in FIGS. 7C-7G. Such induced curvature of cannula (130, 430) by first distal curved portion (965) may limit the impact of any movement of body (110) of instrument (100) on the position of distal tip (952), and may thereby inhibit inadvertent movement of distal tip (952) that might otherwise result from movement of body (110). For example, first distal curved portion (965) and/or the portion of cannula (130, 430) in which first distal curved portion (965) is disposed may effectively absorb some or all of such movement instead of transmitting such movement distally to second distal curved portion (966) and distal straight portion (968) of needle (950). In addition, or alternatively, the induced curvature of cannula (130, 430) by first distal curved portion (965) may further assist in the portion of cannula (130, 430) within the eye (301) conforming to the curvature of the eye (301).

Second distal curved portion (966) may be configured to orient distal tip (952) along one or more predetermined exit axes during distal advancement of needle (950) relative to cannula (130, 430) to protrude from opening (134). For example, distal curved portion (966) may be configured and operable in accordance with at least some of the teachings of U.S. Pat. No. 10,478,553, the disclosure of which is incorporated by reference herein, in its entirety.

F. Example of a Needle with Proximal Curved Portion and First and Second Distal Curved Portions

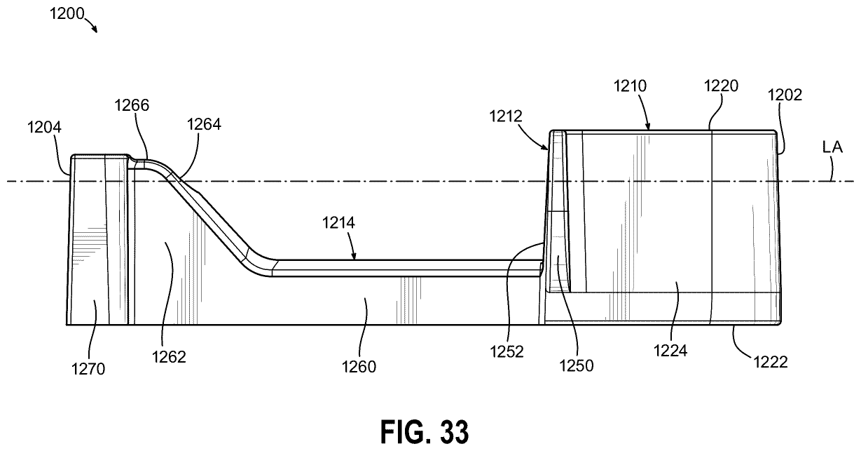

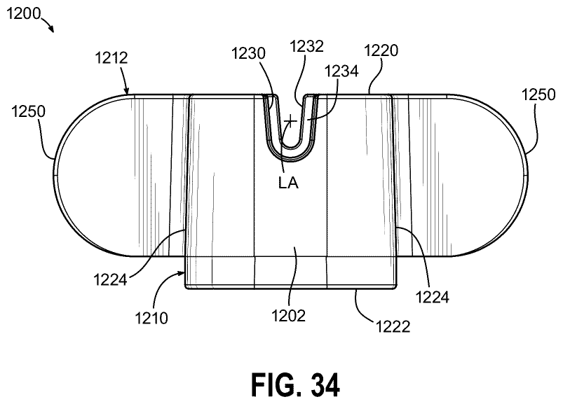

FIGS. 27-28 show another example of a needle (1050) that may be readily incorporated into instrument (100) in place of needle (150). Needle (1050) may be similar to needle (550) and/or needle (950) described above, except as otherwise described below. In this regard, needle (1050) of the present example has a sharp distal tip (1052) and defines a lumen (not shown).

Needle (1050) of the present example includes a substantially straight proximal portion (1060), a substantially curved proximal portion (1062), a substantially straight medial portion (1064), a first substantially curved distal portion (1065), a second substantially curved distal portion (1066), and a substantially straight distal portion (1068). Proximal curved portion (1062) is longitudinally interposed between proximal straight portion (1060) and medial straight portion (1064); medial straight portion (1064) is longitudinally interposed between proximal curved portion (1062) and first distal curved portion (1065); first distal curved portion (1065) is longitudinally interposed between medial straight portion (1064) and second distal curved portion (1066); second distal curved portion (1066) is longitudinally interposed between first distal curved portion (1065) and distal straight portion (1068); and distal straight portion (1068) is longitudinally interposed between second distal curved portion (1066) and distal tip (1052). In the example shown, proximal curved portion (1062) and distal curved portions (1065, 1066) are curved in opposite directions from each other to provide needle (1050) with a generally S-shaped configuration. For example, proximal curved portion (1062) curves generally clockwise in the distal direction while distal curved portions (1065, 1066) each curve generally counterclockwise in the distal direction, within the frame of reference of FIGS. 27-28.

Needle (1050) is configured to provide proximal curved portion (1062) and distal curved portions (1065, 1066) as preformed features, such that needle (1050) is resiliently biased to assume the generally S-shaped configuration shown in FIG. 27. In the example shown, proximal straight portion (1060) has a length (L1); proximal curved portion (1062) has a constant radius of curvature (R1) and an arclength (S1); medial straight portion (1064) has a length (L2); first distal curved portion (1065) has a constant radius of curvature (R2) and an arclength (S2); second distal curved portion (1066) has a constant radius of curvature (R3) and an arclength (S3); and distal straight portion (1068) has a length (L3).

By way of example only, length (L1) may range from approximately 19.92 mm to approximately 29.88 mm, or may be more particularly approximately 24.9 mm; radius of curvature (R1) may range from approximately 16 mm to approximately 24 mm, or may be more particularly approximately 20 mm; arclength (S1) may range from approximately 16 mm to approximately 24 mm, or may be more particularly approximately 20 mm; length (L2) may range from approximately 16 mm to approximately 24 mm, or may be more particularly approximately 20 mm; radius of curvature (R2) may range from approximately 13.6 mm to approximately 20.4 mm, or may be more particularly approximately 17 mm; arclength (S2) may range from approximately 8 mm to approximately 12 mm, or may be more particularly approximately 10 mm; radius of curvature (R3) may range from approximately 2.8 mm to approximately 4.2 mm, or may be more particularly approximately 3.5 mm; arclength (S3) may range from approximately 3.36 mm to approximately 5.04 mm, or may be more particularly approximately 4.2 mm; and/or length (L3) may range from approximately 0.56 mm to approximately 0.84 mm, or may be more particularly approximately 0.7 mm.

In some versions, medial straight portion (1064) may be omitted, such that proximal curved portion (1062) may directly interface with first distal curved portion (1065). In addition, or alternatively, proximal straight portion (1060) may have a slight curvature, such that proximal straight portion (1060) may have an arclength (SO). In some versions, the arclengths (S0, S1, S2, S3) of proximal straight portion (1060), proximal curved portion (1062), first distal curved portion (1065), and second distal curved portion (1066) may be selected from the following table, wherein all values are approximate.

| S3 | S2 | S1 | S0 |
|---|---|---|---|
| 2 mm | 18 mm | 20 mm | 5 mm |
| 3 mm | 8 mm | 12 mm | 0 mm |
| 1.5 mm | 8 mm | 12 mm | 0 mm |
| 3 mm | 8 mm | 12 mm | 0 mm |
| 1.5 mm | 8 mm | 12 mm | 0 mm |
| 1.5 mm | 8 mm | 12 mm | 0 mm |

In some versions, proximal curved portion (1062) may cause the portion of cannula (130, 430) external to the eye (301) to curve away from the eye (301) and toward body (110), in a manner similar to that described above in connection with FIG. 14. Such induced curvature of cannula (130, 430) by proximal curved portion (1062) may limit the impact of any movement of body (110) of instrument (100) on the position of distal tip (1052); and may thereby inhibit inadvertent movement of distal tip (1052) that might otherwise result from movement of body (110). For example, proximal curved portion (1062) and/or the portion of cannula (130, 430) in which proximal curved portion (1062) is disposed may effectively absorb some or all of such movement instead of transmitting such movement distally to medial straight portion (1064), first distal curved portion (1065), second distal curved portion (1066), and distal straight portion (1068) of needle (1050). In addition, or alternatively, the induced curvature of cannula (130, 430) by proximal curved portion (1062) may further assist in the portion of cannula (130, 430) within the eye (301) conforming to the curvature of the eye (301).

G. Example of a Needle with First and Second Proximal Curved Portions and First and Second Distal Curved Portions

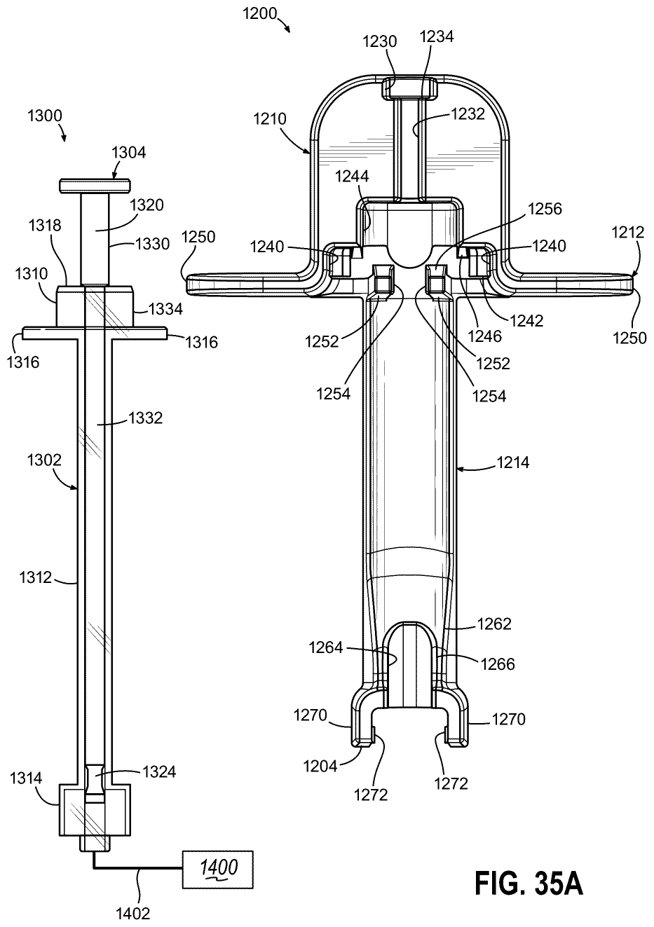

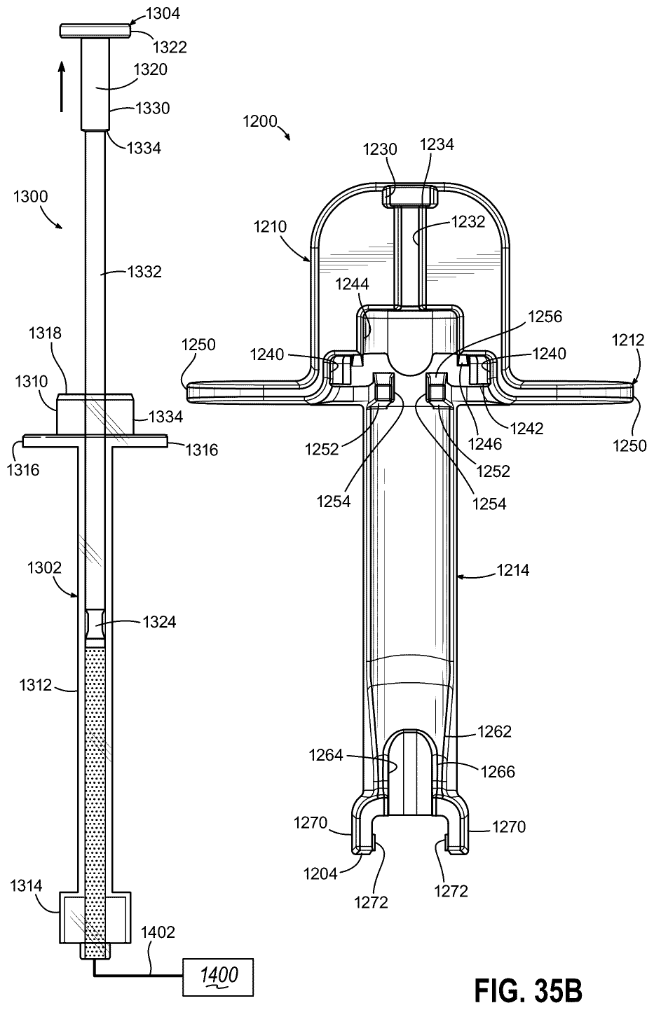

FIGS. 29-30 show another example of a needle (1150) that may be readily incorporated into instrument (100) in place of needle (150). Needle (1150) may be similar to needle (550) and/or needle (950) described above, except as otherwise described below. In this regard, needle (1150) of the present example has a sharp distal tip (1152) and defines a lumen (not shown).

Needle (1150) of the present example includes a substantially straight proximal portion (1160), a first substantially curved proximal portion (1162), a second substantially curved proximal portion (1163), a first substantially curved distal portion (1165), a second substantially curved distal portion (1166), and a substantially straight distal portion (1168). First proximal curved portion (1162) is longitudinally interposed between proximal straight portion (1160) and second proximal curved portion (1163); second proximal curved portion (1163) is longitudinally interposed between first proximal curved portion (1162) and first distal curved portion (1165); first distal curved portion (1165) is longitudinally interposed between second proximal curved portion (1163) and second distal curved portion (1166); second distal curved portion (1166) is longitudinally interposed between first distal curved portion (1165) and distal straight portion (1168); and distal straight portion (1168) is longitudinally interposed between second distal curved portion (1166) and distal tip (1152). In some versions, needle (1150) may include a substantially straight medial portion (not shown) longitudinally interposed between second proximal curved portion (1163) and first distal curved portion (1165), for example. In the example shown, proximal curved portions (1162, 1163) and distal curved portions (1165, 1166) are curved in opposite directions from each other to provide needle (1150) with a generally S-shaped configuration. For example, proximal curved portions (1162, 1163) each curve generally clockwise in the distal direction while distal curved portions (1165, 1066) each curve generally counterclockwise in the distal direction, within the frame of reference of FIGS. 29-30.

Needle (1150) is configured to provide proximal curved portions (1162, 1163) and distal curved portions (1165, 1166) as preformed features, such that needle (1150) is resiliently biased to assume the generally S-shaped configuration shown in FIG. 29. In the example shown, proximal straight portion (1160) has a length (L1); first proximal curved portion (1162) has a constant radius of curvature (R1) and an arclength (S1); second proximal curved portion (1163) has a constant radius of curvature (R2) and an arclength (S2); first distal curved portion (1165) has a constant radius of curvature (R3) and an arclength (S3); second distal curved portion (1166) has a constant radius of curvature (R4) and an arclength (S4); and distal straight portion (1168) has a length (L2).

By way of example only, length (L1) may range from approximately 12.08 mm to approximately 18.12 mm, or may be more particularly approximately 15.1 mm; radius of curvature (R1) may range from approximately 14.4 mm to approximately 21.6 mm, or may be more particularly approximately 18 mm; arclength (S1) may range from approximately 16 mm to approximately 24 mm, or may be more particularly approximately 20 mm; radius of curvature (R2) may range from approximately 10 mm to approximately 15 mm, or may be more particularly approximately 12.5 mm; arclength (S2) may range from approximately 16 mm to approximately 24 mm, or may be more particularly approximately 20 mm; radius of curvature (R3) may range from approximately 10 mm to approximately 15 mm, or may be more particularly approximately 12.5 mm; arclength (S3) may range from approximately 16 mm to approximately 24 mm, or may be more particularly approximately 20 mm; radius of curvature (R4) may range from approximately 2.8 mm to approximately 4.2 mm, or may be more particularly approximately 3.5 mm; arclength (S4) may range from approximately 3.36 mm to approximately 5.04 mm, or may be more particularly approximately 4.2 mm; and/or length (L2) may range from approximately 0.56 mm to approximately 0.84 mm, or may be more particularly approximately 0.7 mm.

In some versions, one or both proximal curved portions (1162, 1163) may cause the portion of cannula (130, 430) external to the eye (301) to curve away from the eye (301) and toward body (110), in a manner similar to that described above in connection with FIG. 14. Such induced curvature of cannula (130, 430) by one or both proximal curved portions (1162, 1163) may limit the impact of any movement of body (110) of instrument (100) on the position of distal tip (1152); and may thereby inhibit inadvertent movement of distal tip (1152) that might otherwise result from movement of body (110). For example, one or both proximal curved portions (1162, 1163) and/or the portion of cannula (130, 430) in which one or both proximal curved portions (1162, 1163) is disposed may effectively absorb some or all of such movement instead of transmitting such movement distally to first distal curved portion (1165), second distal curved portion (1166), and distal straight portion (1168) of needle (1150). In addition, or alternatively, the induced curvature of cannula (130, 430) by one or both proximal curved portions (1162, 1163) may further assist in the portion of cannula (130, 430) within the eye (301) conforming to the curvature of the eye (301).

### V. EXAMPLE OF DOSE DOCK FOR SYRINGE

As mentioned above, bleb fluid source (82) and/or therapeutic agent fluid source (84) of fluid delivery system (80) may comprise a syringe. In some instances, it may be desirable to provide priming of conduit assembly (140) and instrument (100) to purge any air from the fluid path between a barrel of such a syringe and the distal end of needle (150, 550, 650, 750, 850, 950, 1050, 1150), such as prior to insertion of cannula (130, 430) into the patient's eye (301). In this regard, those skilled in the art will recognize that insufficient priming of conduit assembly (140) and instrument (100) may result in some residual air remaining in the fluid path between the syringe barrel and the distal end of needle (150, 550, 650, 750, 850, 950, 1050, 1150), which may then be inadvertently delivered to the subretinal space of the eye (301); and that excessive priming of conduit assembly (140) and instrument (100) may result in a portion of the bleb fluid or therapeutic agent fluid being inadvertently expelled from the distal end of needle (150, 550, 650, 750, 850, 950, 1050, 1150), such that the amount of bleb fluid or therapeutic agent fluid that is ultimately delivered to the subretinal space of the eye (301) may be less than the desired amount. Those skilled in the art will recognize that it may be critical to ensure that an appropriate amount of fluid is delivered to the subretinal space of the eye (301), particularly in view of the anatomical constraints of the subretinal space. In the case of leading bleb (340), delivering too little fluid may result in insufficient separation of the retina (308) from the choroid (306), which may result in inadequate efficacy of subsequently delivered therapeutic agent (342). Similarly, delivering too little therapeutic agent (342) may fail to yield the desired therapeutic effect.

While many surgeons and their assistants may have substantial skill in ensuring that conduit assembly (140) and instrument (100) are appropriately primed before use of the syringe to deliver the fluid, it may be desirable to provide a device that reliably and consistently ensures that conduit assembly (140) and instrument (100) are appropriately primed before use of the syringe to deliver the fluid, such that a patient need not rely so much on the personal skill of the surgeon, etc. to appropriately prime conduit assembly (140) and instrument (100). The following describes an example of a dose dock (1200) that may be used with a conventional syringe, such as that comprising bleb fluid source (82) and/or therapeutic agent fluid source (84) of fluid delivery system (80), to consistently provide precision and accuracy in the amount of air that is purged from the fluid path between a barrel of such a syringe and the distal end of needle (150, 550, 650, 750, 850, 950, 1050, 1150); which may in turn consistently provide precision and accuracy in the amount of fluid that is delivered to the subretinal space as part of the procedure described above with reference to FIGS. 6-7G. While dose dock (1200) is described below for use with instrument (100), it will be appreciated that dose dock (1200) may be used with any other suitable type of instrument, such as any other suitable instrument for subretinal and/or suprachoroidal delivery of a therapeutic agent, and/or any instrument from which a precise and repeatable fluid delivery is desired. For example, dose dock (1200) may be used with a suprachoroidal delivery instrument that is configured and operable in accordance with at least some of the teachings of International Pub. No. WO 2022/172086, entitled “Suprachoroidal Injection Device,” published Aug. 18, 2022, the disclosure of which is incorporated by reference herein, in its entirety.

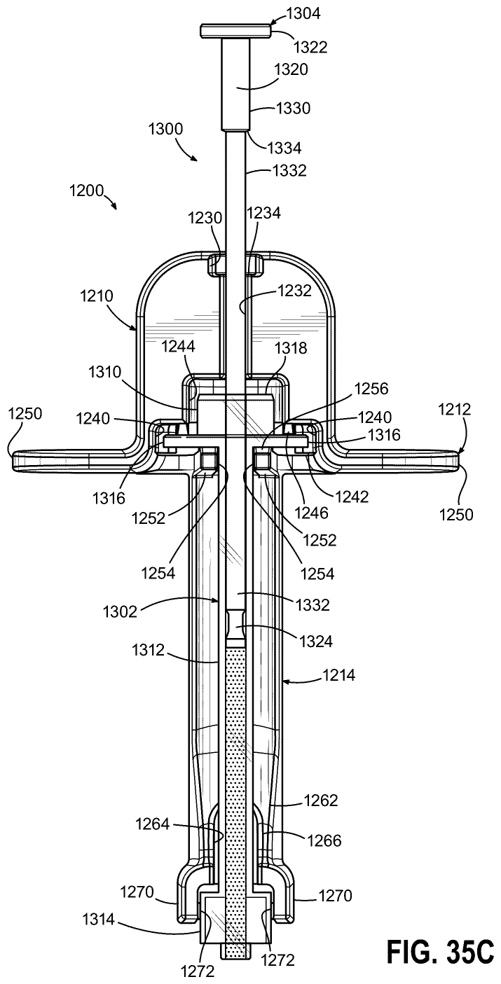

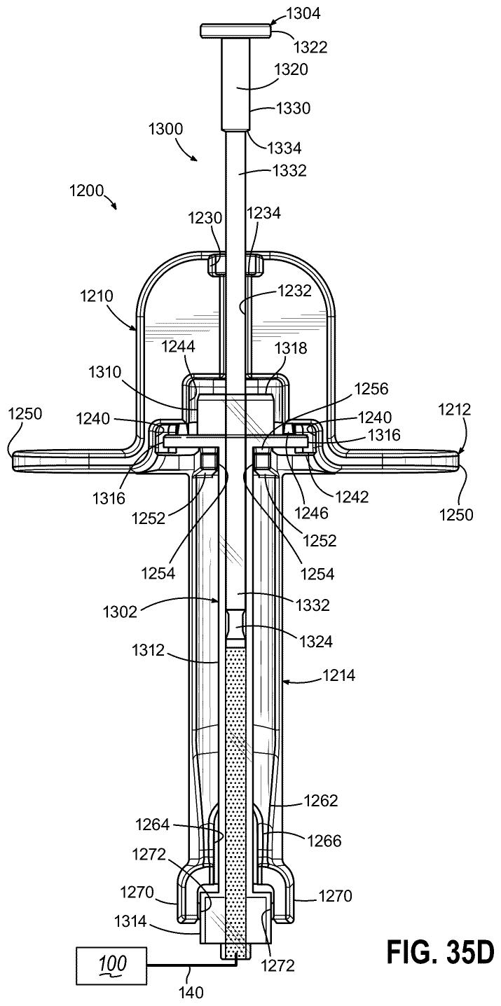

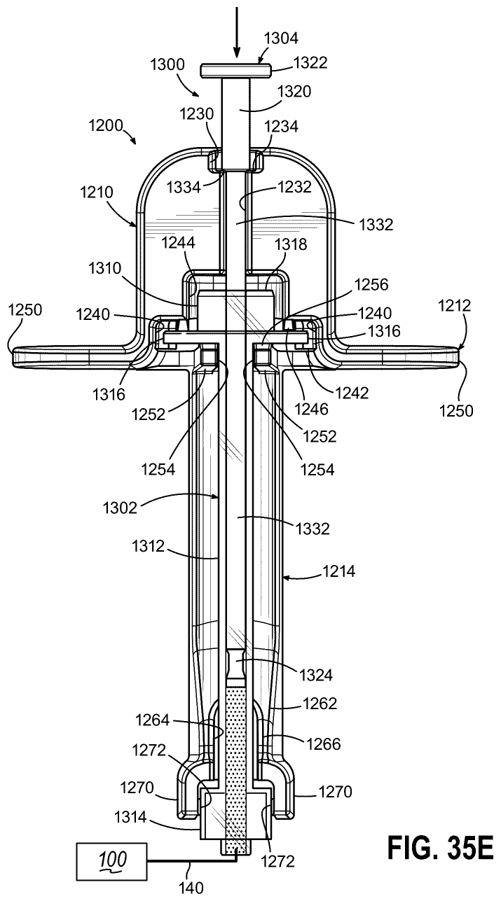

As shown in FIGS. 31-34, dose dock (1200) of the present example extends between a proximal end (1202) and a distal end (1204) along a longitudinal axis (LA) (FIGS. 32-34); and includes a proximal main body portion (1210), an intermediate hilt portion (1212), and a distal support portion (1214). More particularly, main body portion (1210) extends distally from proximal end (1202) to hilt portion (1212), which extends distally from main body portion (1210) to support portion (1214), which extends distally from hilt portion (1212) to distal end (1204). In the example shown, main body portion (1210), hilt portion (1212), and support portion (1214) are integrally formed together with each other as a unitary (e.g., monolithic) piece to define dose dock (1200). In this regard, dose dock (1200) may be manufactured via 3D printing, injection molding, investment casting, machining, and/or any other suitable manufacturing techniques. It will be appreciated that dose dock (1200) may be constructed of any suitable material, such as a polymeric material (e.g., plastic) or a metallic material. In some versions, dose dock (1200) may be constructed of a thermoplastic, such as a polycarbonate.

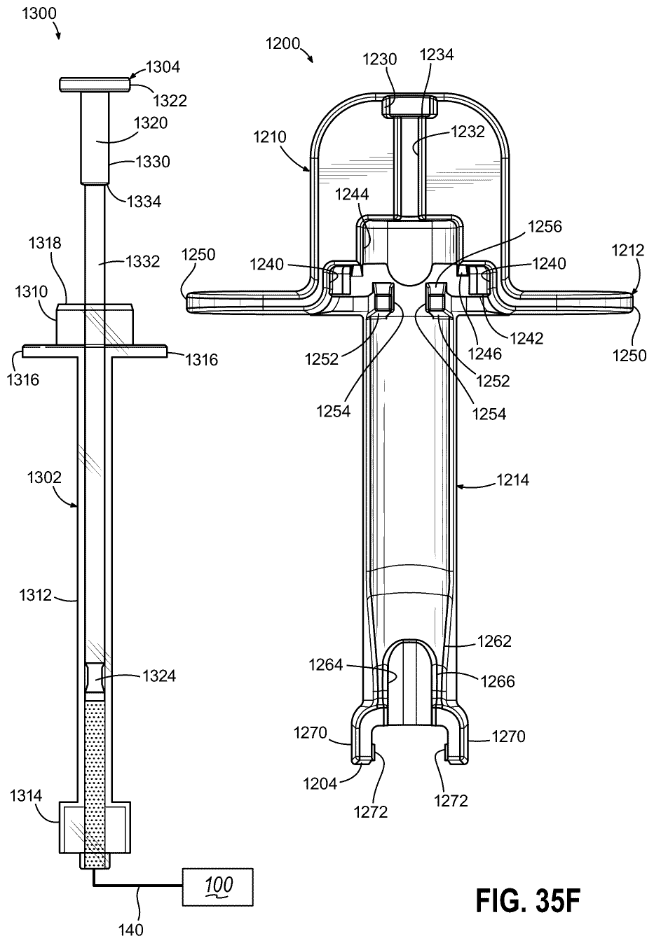

Main body portion (1210) of the present example includes a top surface (1220), a bottom surface (1222), and a pair of side surfaces (1224). In the example shown, a generally U-shaped proximal recess (1230) extends distally from proximal end (1202) along longitudinal axis (LA) and downwardly from top surface (1220), and a generally U-shaped proximal channel (1232) extends distally from proximal recess (1230) along longitudinal axis (LA) and downwardly from top surface (1220). Proximal channel (1232) is sized and configured to slidably receive a portion of a syringe plunger rod (e.g., distal portion (1332) of plunger rod (1320)), as described in greater detail below. As best shown in FIG. 34, proximal recess (1230) and proximal channel (1232) may be similarly shaped, with proximal recess (1230) being wider than proximal channel (1232) in a lateral direction and/or deeper than proximal channel (1232) relative to top surface (1220). In this manner, proximal recess (1230) defines a generally U-shaped, proximally-facing stop surface (1234) that is configured to selectively engage (e.g., abut) a shoulder of a syringe plunger (e.g., shoulder (1334) of plunger (1304)) to arrest distal advancement of the syringe plunger, as described in greater detail below.

In the example shown, a pair of distal recesses (1240) extend proximally from a distal end of main body portion (1210) and downwardly from top surface (1220) to respective lower ledges (1242) on opposite sides of longitudinal axis (LA), and a generally U-shaped intermediate recess (1244) extends proximally from distal recesses (1240) along longitudinal axis (LA) to proximal channel (1232). Distal recesses (1240) are sized and configured to receive respective syringe finger flanges (e.g., finger flanges (1316)), as described in greater detail below. As shown, a pair of tabs (1246) extend distally from respective proximal ends of distal recesses (1240) on opposite sides of longitudinal axis (LA); and may be configured to engage the respective syringe finger flanges to assist with limiting longitudinal movement of the syringe finger flanges relative to dose dock (1200). Intermediate recess (1244) is sized and configured to receive a portion of a syringe barrel (e.g., proximal cylindrical portion (1310) of syringe barrel (1302)), as described in greater detail below.

Hilt portion (1212) of the present example includes a pair of finger flanges (1250) extending laterally outwardly relative to side surfaces (1224) of main body portion (1210) on opposite sides of longitudinal axis (LA). Finger flanges (1250) may be configured to extend outwardly in the same directions as corresponding finger flanges of a syringe barrel coupled to dose dock (1200) (e.g., finger flanges (1316) of syringe barrel (1302)), such that finger flanges (1250) may be configured to be gripped by the operator in a manner similar to that with which the operator might otherwise directly grip the syringe finger flanges.

In the example shown, hilt portion (1212) also includes a pair of upwardly-extending deflectable beams (1252) on opposite sides of longitudinal axis (LA) that are configured to cooperate with each other to securely grip a portion of a syringe barrel (e.g., intermediate cylindrical portion (1312) of syringe barrel (1302)), as described in greater detail below. In this regard, a pair of detents (1254) extend laterally inwardly from respective upper ends of deflectable beams (1252); and may be configured to frictionally engage upper and/or side surfaces of the syringe barrel to inhibit movement of the syringe barrel in an upward direction relative to dose dock (1200). In some versions, deflectable beams (1252) may be resiliently biased toward an undeflected state and may be configured to flex to one or more deflected states. In addition, or alternatively, deflectable beams (1252) may be configured to provide a snap-fit engagement with the syringe barrel. For example, deflectable beams (1252) may be configured to flex laterally outwardly away from each other when the syringe barrel is above detents (1254) in response to a threshold downwardly-directed force being applied to the syringe barrel to permit insertion of the syringe barrel between and/or under detents (1254); and to then resiliently return to the respective undeflected states to frictionally engage the upper and/or side surfaces of the syringe barrel. Deflectable beams (1252) may be configured to similarly flex laterally outwardly away from each other when the syringe barrel is between and/or under detents (1254) in response to a threshold upwardly-directed force being applied to the syringe barrel to permit removal of the syringe barrel from dose dock (1200). As shown, a pair of tabs (1256) extend proximally from respective upper ends of deflectable beams (1252), and may be configured to engage corresponding finger flanges of a syringe barrel coupled to dose dock (1200) (e.g., finger flanges (1316) of syringe barrel (1302)) to assist with limiting longitudinal movement of the syringe finger flanges relative to dose dock (1200), such as by capturing the respective syringe finger flanges against the respective tabs (1246) of main body portion (1210) and/or against the respective ledges (1242) of main body portion (1210). In some versions, ledges (1242), tabs (1246), and/or tabs (1256) may be configured to cooperate with each other to promote positioning a portion of the syringe barrel (e.g., stop surface (1318) of syringe barrel (1302)) at a predetermined location relative to stop surface (1234).

Support portion (1214) of the present example includes an elongate arm (1260) extending distally from a lower region of hilt portion (1212) below deflectable beams (1252), and a cradle (1262) extending generally upwardly from a distal end of arm (1260), such that support portion (1214) is generally L-shaped. In the example shown, a generally U-shaped distal channel (1264) extends downwardly from a top surface (1266) of cradle (1262) and longitudinally along longitudinal axis (LA), such that distal channel (1264) is longitudinally aligned with proximal channel (1232). Distal channel (1264) is sized and configured to receive a portion of syringe barrel (e.g., intermediate cylindrical portion (1312) of syringe barrel (1302)), as described in greater detail below.

In the example shown, support portion (1214) also includes a pair of distally-extending deflectable flanges (1270) on opposite sides of longitudinal axis (LA) that are configured to cooperate with each other to securely grip a portion of the syringe barrel (e.g., distal cylindrical portion (1314) of syringe barrel (1302)), as described in greater detail below. In this regard, a pair of detents (1272) extend laterally inwardly from respective upper ends of deflectable flanges (1270); and may be configured to frictionally engage upper and/or side surfaces of the syringe barrel to inhibit movement of the syringe barrel in an upward direction relative to dose dock (1200). In some versions, deflectable flanges (1270) may be resiliently biased toward an undeflected state and may be configured to flex to one or more deflected states. In addition, or alternatively, deflectable flanges (1270) may be configured to provide a snap-fit engagement with the syringe barrel. For example, deflectable flanges (1270) may be configured to flex laterally outwardly away from each other when the syringe barrel is above detents (1272) in response to a threshold downwardly-directed force being applied to the syringe barrel to permit insertion of the syringe barrel between and/or under detents (1272); and to then resiliently return to the respective undeflected states to frictionally engage the upper and/or side surfaces of the syringe barrel. Deflectable flanges (1270) may be configured to similarly flex laterally outwardly away from each other when the syringe barrel is between and/or under detents (1272) in response to a threshold upwardly-directed force being applied to the syringe barrel to permit removal of the syringe barrel from dose dock (1200).

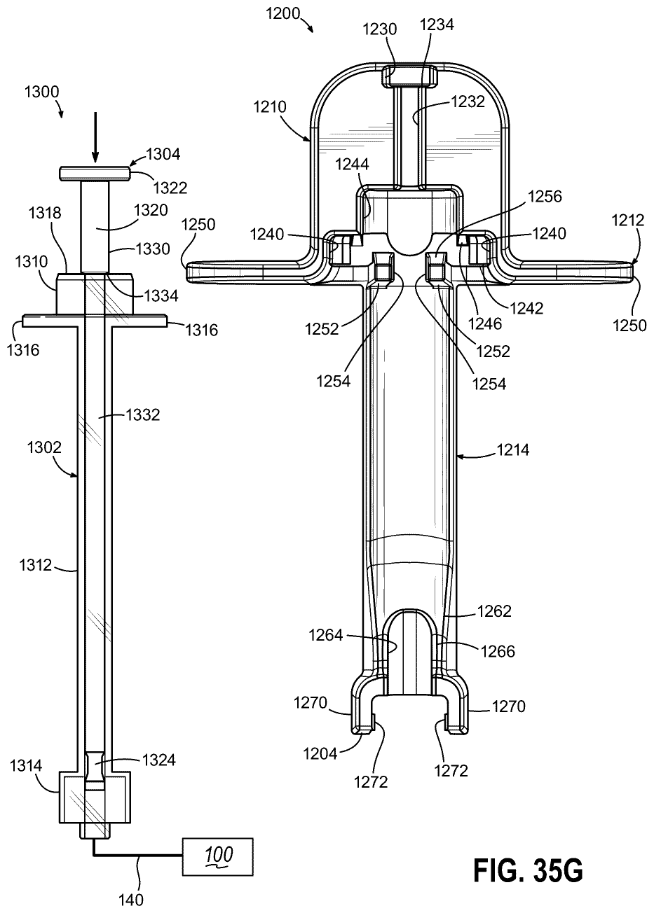

FIGS. 35A-35G show an example of dose dock (1200) being used with an example of a syringe (1300). Syringe (1300) of this example includes a barrel (1302) and a plunger (1304). Barrel (1302) includes a proximal cylindrical portion (1310), an intermediate cylindrical portion (1312), and a distal cylindrical portion (1314). Barrel (1302) also includes a pair of finger flanges (1316) extending laterally outwardly relative to proximal cylindrical portion (1310). In the example shown, proximal cylindrical portion (1310) defines a generally annular, proximally-facing stop surface (1318) that is configured to selectively engage (e.g., abut) a portion of plunger (1304) to arrest distal advancement of plunger (1304), as described in greater detail below. In this regard, plunger (1304) includes an elongate rod (1320), a thumb flange (1322) at a proximal end of rod (1320), and a piston (1324) at a distal end of rod (1320). Piston (1324) is sealingly disposed in barrel (1302), such that the longitudinal position of piston (1324) within barrel (1302) may be varied to selectively vary the effective internal capacity of barrel (1302), which will in turn influence the volume of fluid contained within barrel (1302). In the example shown, rod (1320) includes a relatively wide proximal portion (1330) and a relatively narrow distal portion (1332) to define a generally annular, distally-facing shoulder (1334) that is configured to selectively engage (e.g., abut) stop surface (1318) of barrel (1302) and stop surface (1234) of dose dock (1200). In some versions, syringe (1300) includes the 0.2 mL Zero Residual syringe by SJJ Solutions of Zuid Holland, the Netherlands. In one aspect, syringe (1300) is as described in U.S. Pat. No. D961,067 S1, entitled “Syringe,” issued Aug. 16, 2022, or International Pub. No. WO 2020/013692 A1, entitled “Needle Hub and Syringe Arrangement,” published Jan. 16, 2020, each of which is incorporated by reference in its entirety. It will be appreciated that syringe (1300) may include any other suitable type of syringe from any other suitable source.

As shown in FIG. 35A, barrel (1302) is initially coupled with a fluid source (1400) via a conduit (1402). In some instances, fluid source (1400) contains leading bleb (340) fluid. In some other instances, fluid source (1400) contains therapeutic agent (342) fluid. When barrel (1302) is initially coupled with fluid source (1400), plunger (1304) is in a distal position relative to barrel (1302). Also, when barrel (1302) is initially coupled with fluid source (1400), syringe (1300) is completely decoupled from dose dock (1200) in this example.

With barrel (1302) sufficiently coupled with fluid source (1400), the operator retracts plunger (1304) proximally relative to barrel (1302) to the position shown in FIG. 35B. During such proximal retraction of plunger (1304), syringe (1300) draws a volume of fluid from fluid source (1400) into barrel (1302). This volume may be greater than the volume of fluid that will ultimately be delivered to the subretinal space of the eye (301).

Once a volume of fluid has been drawn from fluid source (1400) into barrel (1302), syringe (1300) is decoupled from fluid source (1400) and coupled with dose dock (1200). In particular, as shown in FIG. 35C, distal portion (1332) of rod (1320) is inserted into proximal channel (1232) of dose dock (1200); proximal cylindrical portion (1310) of barrel (1302) is inserted into intermediate recess (1244) of dose dock (1200); finger flanges (1316) of barrel (1302) are inserted into the respective distal recesses (1240) of dose dock (1200); intermediate cylindrical portion (1312) of barrel (1302) is inserted into distal channel (1264) of dose dock (1200) and securely gripped by deflectable beams (1252) of dose dock (1200); and distal cylindrical portion (1314) of barrel (1302) is securely gripped by deflectable flanges (1270) of dose dock (1200). In this manner, recesses (1240, 1244), distal channel (1264), deflectable beams (1252), and deflectable flanges (1270) may collectively define a receptacle that is configured to securely receive barrel (1302). In some versions, any one or more of recesses (1240, 1244), distal channel (1264), deflectable beams (1252), and/or deflectable flanges (1270) are configured to provide a snap fit with the corresponding portion(s) of barrel (1302) to thereby assist with securing barrel (1302) to dose dock (1200). In addition, or in the alternative, any one or more of recesses (1240, 1244), distal channel (1264), deflectable beams (1252), and/or deflectable flanges (1270) may include one or more elastomeric features and/or other features that that are configured to grip or otherwise retain the corresponding portion(s) of barrel (1302). In the present example, at the stage shown in FIG. 35C, plunger (1304) is still in the proximal position relative to barrel (1302).

Next, as shown in FIG. 35D, barrel (1302) is coupled with instrument (100) via conduit assembly (140), both of which are described above. It will be appreciated that instrument (100) may include any suitable cannula, such as any of cannulas (130, 430) described above, and/or may include any suitable needle, such as any of needles (150, 550, 650, 750, 850, 950, 1050, 1150) described above.

After reaching the state shown in FIG. 35D, the operator may advance plunger (1304) distally relative to barrel (1302) until reaching the stage shown in FIG. 35E. At this stage, shoulder (1334) of plunger (1304) abuts stop surface (1234) of dose dock (1200). During the transition from the state shown in FIG. 35D to the state shown in FIG. 35E, plunger (1304) drives a portion of the fluid from barrel (1302). This may in turn provide priming of conduit assembly (140) and instrument (100), purging any air from the fluid path between barrel (1302) and the distal end of needle (150, 550, 650, 750, 850, 950, 1050, 1150). Thus, during the transition from the state shown in FIG. 35D to the state shown in FIG. 35E, cannula (130, 430) may not yet be inserted into the patient's eye (301). The fluid expelled from the distal end of needle (150, 550, 650, 750, 850, 950, 1050, 1150) during the transition from the state shown in FIG. 35D to the state shown in FIG. 35E may be directed into an appropriate waste receptacle.

In some versions, the volume of the fluid expelled from the distal end of needle (150, 550, 650, 750, 850, 950, 1050, 1150) during the transition from the state shown in FIG. 35D to the state shown in FIG. 35E may be generally equal to the difference between the volume of the fluid drawn by syringe (1300) from fluid source (1400) into barrel (1302) during the transition from the state shown in FIG. 35A to the state shown in FIG. 35B, and a predetermined volume of the fluid that is desired to be ultimately delivered to the subretinal space of the eye (301). For example, the volume of the fluid expelled from the distal end of needle (150, 550, 650, 750, 850, 950, 1050, 1150) during the transition from the state shown in FIG. 35D to the state shown in FIG. 35E may be approximately 250 microliters in cases where the predetermined volume of the fluid that is desired to be ultimately delivered to the subretinal space of the eye (301) is approximately 100 microliters but approximately 350 microliters of the fluid were drawn by syringe (1300) from fluid source (1400) into barrel (1302) during the transition from the state shown in FIG. 35A to the state shown in FIG. 35B. As another example, the volume of the fluid expelled from the distal end of needle (150, 550, 650, 750, 850, 950, 1050, 1150) during the transition from the state shown in FIG. 35D to the state shown in FIG. 35E may be approximately 100 microliters in cases where the predetermined volume of the fluid that is desired to be ultimately delivered to the subretinal space of the eye (301) is approximately 100 microliters but approximately 200 microliters of the fluid were drawn by syringe (1300) from fluid source (1400) into barrel (1302) during the transition from the state shown in FIG. 35A to the state shown in FIG. 35B. As yet another example, the volume of the fluid expelled from the distal end of needle (150, 550, 650, 750, 850, 950, 1050, 1150) during the transition from the state shown in FIG. 35D to the state shown in FIG. 35E may be approximately 15 microliters in cases where the predetermined volume of the fluid that is desired to be ultimately delivered to the subretinal space of the eye (301) is approximately 100 microliters but approximately 115 microliters of the fluid were drawn by syringe (1300) from fluid source (1400) into barrel (1302) during the transition from the state shown in FIG. 35A to the state shown in FIG. 35B.

In addition, or alternatively, the volume of the fluid expelled from the distal end of needle (150, 550, 650, 750, 850, 950, 1050, 1150) during the transition from the state shown in FIG. 35D to the state shown in FIG. 35E may be greater than or generally equal to the dead volume of the fluid path between barrel (1302) and the distal end of needle (150, 550, 650, 750, 850, 950, 1050, 1150). For example, the volume of the fluid expelled from the distal end of needle (150, 550, 650, 750, 850, 950, 1050, 1150) during the transition from the state shown in FIG. 35D to the state shown in FIG. 35E may be at least approximately 80 microliters in cases where the dead volume is approximately 80 microliters.

With shoulder (1334) of plunger (1304) abutting stop surface (1234) of dose dock (1200) as shown in FIG. 35E, plunger (1304) may not advance distally relative to barrel (1302). By preventing plunger (1304) from translating distally relative to barrel (1302), shoulder (1334) and stop surface (1234) prevent the inadvertent expulsion of fluid from barrel (1302); thereby preventing the inadvertent expulsion of fluid from needle (150, 550, 650, 750, 850, 950, 1050, 1150) to maintain a predetermined volume of fluid in barrel (1302) and the entire fluid path between barrel (1302) and the distal end of needle (150, 550, 650, 750, 850, 950, 1050, 1150). In some versions, the arresting of distal translation of plunger (1304) relative to barrel (1302) caused by shoulder (1334) abutting stop surface (1234) may provide a positive indication to the operator that conduit assembly (140) and instrument (100) have been appropriately primed, with any air having been purged from the fluid path between barrel (1302) and the distal end of needle (150, 550, 650, 750, 850, 950, 1050, 1150) during the transition from the state shown in FIG. 35D to the state shown in FIG. 35E; and that barrel (1302) contains the appropriate predetermined amount of fluid. For example, the predetermined amount of fluid contained within barrel (1302) when in the state shown in FIG. 35E may correspond to a desired predetermined volume of fluid for subsequent delivery to form leading bleb (340). By way of example only, the predetermined amount of fluid contained within barrel (1302) may range from approximately 50 microliters to approximately 300 microliters. In some versions, the predetermined amount of fluid contained within barrel (1302) is approximately 100 microliters. As another example, the predetermined amount of fluid contained within barrel (1302) may range from approximately 50 microliters to approximately 250 microliters. As yet another example, the predetermined amount of fluid contained within barrel (1302) may range from approximately 50 microliters to approximately 200 microliters. In this regard, stop surface (1234) may be longitudinally disposed at a predetermined position relative to one or more portions of syringe (1300), instrument (100), and/or conduit (140), with such a predetermined position being selected to ensure that any air within the fluid path between barrel (1302) and the distal end of needle (150, 550, 650, 750, 850, 950, 1050, 1150) is purged during the transition from the state shown in FIG. 35D to the state shown in FIG. 35E.

After achieving the state shown in FIG. 35E, the operator may complete the steps shown in FIGS. 7A-7E and described above. In this example, barrel (1302) contains the fluid to form leading bleb (340). In order to ready syringe (1300) for delivery of leading bleb (340), the operator may remove syringe (1300) from dose dock (1200) such that shoulder (1334) may be disengaged from stop surface (1234) of dose dock (1200), as shown in FIG. 35F. Thus, with shoulder (1334) disengaged from stop surface (1234) of dose dock (1200), plunger (1304) is free to advance distally relative to barrel (1302) from the longitudinal position shown in FIG. 35F. In some cases, the operator refrains from removing syringe (1300) from dose dock (1200) until after reaching the stage of the procedure shown in FIG. 7E. In some other cases, the operator removes syringe (1300) from dose dock (1200) prior to inserting any portion of instrument (100) into the patient's eye (301). For example, in instances where dose dock (1200) is used with a suprachoroidal delivery instrument that is configured and operable in accordance with at least some of the teachings of International Pub. No. WO 2022/172086, the operator may remove syringe (1300) from dose dock (1200) prior to placing a needle of such an instrument on the sclera (304).

Once needle (150, 550, 650, 750, 850, 950, 1050, 1150) has traversed the choroid (306) and is positioned to deliver fluid to the subretinal space as shown in FIG. 7E, and syringe (1300) has been removed from dose dock (1200) as shown in FIG. 35F, the operator may advance plunger (1304) distally relative to barrel (1302) until shoulder (1334) of plunger (1304) engages stop surface (1318) of barrel (1302). This state is shown in FIG. 35G. As plunger (1304) travels from the longitudinal position shown in FIG. 35F to the longitudinal position shown in FIG. 35G, plunger (1304) drives a predetermined volume of fluid from barrel (1302). For example, the predetermined volume of fluid driven from barrel (1302) during the transition from the state shown in FIG. 35F to the state shown in FIG. 35G may correspond to a desired predetermined volume of fluid for delivery to form leading bleb (340). By way of example only, the predetermined volume of fluid driven from barrel (1302) may range from approximately 50 microliters to approximately 300 microliters. As another example, the predetermined volume of fluid driven from barrel (1302) may range from approximately 50 microliters to approximately 250 microliters. As yet another example, the predetermined volume of fluid driven from barrel (1302) may range from approximately 50 microliters to approximately 200 microliters. In some versions, the predetermined volume of fluid driven from barrel (1302) is approximately 100 microliters. This provides communication of fluid through conduit assembly (140) and associated components of instrument (100), ultimately resulting in delivery of leading bleb (340) as shown in FIG. 7F. The volume of fluid forming leading bleb (340) will be dictated by the vertical distance between stop surface (1234) of dose dock (1200) and stop surface (1318) of barrel (1302). Since this vertical distance between stop surfaces (1234, 1318) may be fixed and predetermined (e.g., based on where stop surface (1318) is located when syringe (1300) is coupled to dose dock (1200)), dose dock (1200) will ensure that syringe (1300) predictably delivers a precise, predetermined volume of fluid for leading bleb (340) on a consistent basis. By way of example only, the predetermined volume of fluid delivered for leading bleb (340) may range from approximately 50 microliters to approximately 300 microliters. As another example, the predetermined volume of fluid delivered for leading bleb (340) may range from approximately 50 microliters to approximately 250 microliters. As yet another example, the predetermined volume of fluid delivered for leading bleb (340) may range from approximately 50 microliters to approximately 200 microliters. In some versions, the predetermined volume of fluid for leading bleb (340) is approximately 100 microliters.

In some instances, a combination of a second dose dock (1200) and a second syringe (1300) is also in fluid communication with conduit assembly (140), with the second syringe (1300) containing therapeutic agent (342). As noted above, both syringes (1300) may be coupled with conduit assembly (140) via a “Y” fitting or any other suitable component(s) as will be apparent to those skilled in the art in view of the teachings herein. In such scenarios, the combination of the second dose dock (1200) and second syringe (1300) may undergo the same steps described above with reference to FIGS. 35A-35G. However, the plunger (1304) of the second syringe (1300) would not be advanced from the position shown in FIG. 35F to the position shown in FIG. 35G until after the plunger (1304) of the first syringe (1300) had been advanced from the position shown in FIG. 35F to the position shown in FIG. 35G to thereby deliver leading bleb (340). In other words, the plunger (1304) of the second syringe (1300) would not be advanced from the position shown in FIG. 35F to the position shown in FIG. 35G to deliver therapeutic agent (342) to the subretinal space as shown in FIG. 7G until after leading bleb (340) had already been delivered as shown in FIG. 7F.

In cases where two separate combinations of a dose dock (1200) and syringe (1300) are used—one for leading bleb (340) fluid and the other for therapeutic agent (342) fluid—the corresponding dose docks (1200) may have slightly different main body portions (1210) to provide different predetermined volumes of delivered fluid. For instance, main body portion (1210) that is used with the syringe (1300) containing fluid for leading bleb (340) may be configured to provide delivery of a first predetermined volume of fluid; while main body portion (1210) that is used with the syringe (1300) containing therapeutic agent (342) may be configured to provide delivery of a second predetermined volume of fluid. These different main body portions (1210) may vary based on the longitudinal position of their corresponding stop surfaces (1234). In other words, different dose docks (1200) may vary based on the longitudinal distance between the respective stop surface (1234) and the stop surface (1318) of the corresponding syringe (1300).

While the foregoing example provides use of the combination of dose dock (1200) and syringe (1300) in an ocular procedure, the combination of dose dock (1200) and syringe (1300) may be used in various other kinds of medical procedures involving other regions of a patient's anatomy other than the eye (301). It is therefore contemplated that the present invention is not necessarily limited to use in the ocular procedure shown in FIGS. 7A-7G and described above. Various other suitable scenarios in which dose dock (1200) may be used will be apparent to those skilled in the art in view of the teachings herein.

### VI. EXEMPLARY COMBINATIONS

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus, comprising: (a) a body; (b) a cannula extending distally from the body, wherein the cannula is flexible, wherein the cannula is sized and configured to advance between a sclera and a choroid of a patient's eye; and (c) a needle slidably disposed in the cannula, wherein the needle includes: (i) a sharp distal tip, wherein the needle is configured to translate relative to the cannula between a proximal position and a distal position, wherein the distal tip is configured to be positioned inside the cannula when the needle is in the proximal position, wherein the distal tip is configured to be positioned outside the cannula when the needle is in the distal position, (ii) at least one proximal curved portion, wherein the needle is resiliently biased to extend along at least one proximal curve through the at least one proximal curved portion, and (iii) at least one distal curved portion, wherein the needle is resiliently biased to extend along at least one distal curve through the at least one distal curved portion, wherein the at least one distal curve is different from the at least one proximal curve.

### Example 2

The apparatus of Example 1, wherein the needle further includes a distal straight portion extending along an exit axis, wherein the distal straight portion is longitudinally interposed between the at least one distal curved portion and the sharp distal tip, wherein the needle is resiliently biased to extend along a straight path along the distal straight portion.

### Example 3

The apparatus of any of Examples 1 through 2, wherein the needle further includes a proximal straight portion, wherein the at least one proximal curved portion is longitudinally interposed between the proximal straight portion and the at least one distal curved portion, wherein the needle is resiliently biased to extend along a straight path along the proximal straight portion.

### Example 4

The apparatus of any of Examples 1 through 3, wherein the needle further includes a medial straight portion, wherein the medial straight portion is longitudinally interposed between the at least one proximal curved portion and the at least one distal curved portion, wherein the needle is resiliently biased to extend along a straight path along the medial straight portion.

### Example 5

The apparatus of any of Examples 1 through 4, wherein the at least one proximal curved portion includes first and second proximal curved portions, wherein the needle is resiliently biased to extend along first and second proximal curves through the first and second proximal curved portions, respectively.

### Example 6

The apparatus of any of Examples 1 through 5, wherein the at least one distal curved portion includes first and second distal curved portions, wherein the needle is resiliently biased to extend along first and second distal curves through the first and second distal curved portions, respectively.

### Example 7

The apparatus of any of Examples 1 through 6, wherein the at least one proximal curved portion and the at least one distal curved portion are curved opposite directions from each other.

### Example 8

The apparatus of any of Examples 1 through 7, wherein the needle is resiliently biased to define an “S” shape.

### Example 9

The apparatus of any of Examples 1 through 8, wherein the at least one proximal curved portion is configured to be positioned within the cannula when the needle is in the distal position.

### Example 10

The apparatus of any of Examples 1 through 9, wherein the at least one proximal curved portion is configured to be positioned external to the patient's eye when the needle is in the distal position.

### Example 11

The apparatus of any of Examples 1 through 10, wherein the at least one proximal curved portion is configured to inhibit movement of the body from being transmitted to the distal tip.

### Example 12

The apparatus of any of Examples 1 through 11, wherein the at least one proximal curved portion is configured to impart a curvature to the cannula.

### Example 13

The apparatus of any of Examples 1 through 12, wherein the needle comprises nitinol.

### Example 14

The apparatus of any of Examples 1 through 13, wherein the distal tip is beveled.

### Example 15

The apparatus of any of Examples 1 through 14, wherein the cannula is flexible enough to conform to structures and contours of the patient's eye yet the cannula has sufficient column strength to permit advancement of the cannula between the sclera and the choroid of the patient's eye without buckling.

### Example 16

An apparatus, comprising: (a) a body; (b) a cannula extending distally from the body, wherein the cannula is flexible, wherein the cannula is sized and configured to advance between a sclera and a choroid of a patient's eye; and (c) a needle slidably disposed in the cannula, wherein the needle includes: (i) a sharp distal tip, wherein the needle is configured to translate relative to the cannula between a proximal position and a distal position, wherein the distal tip is configured to be positioned inside the cannula when the needle is in the proximal position and wherein the distal tip is configured to be positioned outside the cannula when the needle is in the distal position, (ii) a first curved portion, wherein the needle is resiliently biased to extend along a first curve through the first curved portion, and (iii) a second curved portion longitudinally spaced apart from the first curved portion, wherein the needle is resiliently biased to extend along a second curve through the second curved portion.

### Example 17

An apparatus, comprising: (a) a body; (b) a cannula extending distally from the body, wherein the cannula is flexible, wherein the cannula is sized and configured to advance between a sclera and a choroid of a patient's eye, wherein the cannula includes: (i) a proximal segment having a first cross-sectional area, (ii) a medial segment having a second cross-sectional area, and (iii) a distal segment having a third cross-sectional area greater than the second cross-sectional area; and (c) a needle slidably disposed in the cannula

### Example 18

The apparatus of Example 17, wherein the second cross-sectional area is less than the first cross-sectional area.

### Example 19

The apparatus of any of Examples 17 through 18, wherein the proximal segment has a first cross-sectional shape, wherein the medial segment has a second cross-sectional shape, wherein the distal segment has a third cross-sectional shape different from the second cross-sectional shape.

### Example 20

The apparatus of any of Examples 17 through 19, wherein the cannula further includes a wedge-shaped distal end.

### Example 21

The apparatus of any of Examples 17 through 20, wherein the proximal segment has a first stiffness, wherein the medial segment has a second stiffness different from the first stiffness.

### Example 22

The apparatus of Example 21, wherein the distal segment has a third stiffness different from the second stiffness.

### Example 23

An apparatus, comprising: (a) a body; (b) a cannula extending distally from the body along a length, wherein the cannula is flexible, wherein the cannula is sized and configured to advance between a sclera and a choroid of a patient's eye, wherein the cannula has a varying stiffness along the length; and (c) a needle slidably disposed in the cannula.

### Example 24

The apparatus of Example 23, wherein the cannula includes: (i) a proximal segment having a first stiffness, (ii) a medial segment having a second stiffness different from the first stiffness, and (iii) a distal segment having a third stiffness different from the second stiffness.

### Example 25

An apparatus, comprising: (a) a main body portion including: (i) a proximal channel extending along a longitudinal axis and configured to slidably receive a plunger of a syringe, and (ii) a first stop surface disposed at a fixed location proximal of the proximal channel, the first stop surface facing proximally and being configured to selectively engage the plunger of the syringe to arrest distal advancement of the plunger relative to a barrel of the syringe; (b) first and second deflectable beams disposed distal of the proximal channel and on first and second sides of the longitudinal axis, respectively, the first and second beams being configured to cooperate with each other to securely grip the barrel of the syringe; and (c) first and second deflectable flanges disposed distal of the first and second beams on the first and second sides of the longitudinal axis, respectively, the first and second flanges being configured to cooperate with each other to grip the barrel of the syringe.

### Example 26

The apparatus of Example 25, further comprising a hilt portion disposed distal of the main body portion, the hilt portion including at least one finger flange extending laterally outwardly relative to the longitudinal axis and configured to be gripped by a user.

### Example 27

The apparatus of Example 26, wherein the hilt portion includes the first and second beams.

### Example 28

The apparatus of any of Examples 26 through 27, further comprising a support portion disposed distal of the hilt portion, the support portion including an elongate arm and a cradle at a distal end of the elongate arm.

### Example 29

The apparatus of Example 28, wherein the support portion includes the first and second flanges.

### Example 30

The apparatus of any of Examples 28 through 29, wherein the cradle includes a distal channel extending along the longitudinal axis and configured to receive the barrel of the syringe.

### Example 31

The apparatus of any of Examples 24 through 30, wherein the main body portion further includes a proximal recess disposed proximal of the proximal channel to define the first stop surface.

### Example 32

The apparatus of any of Examples 24 through 31, wherein the main body portion further includes an intermediate recess disposed distal of the proximal channel, the intermediate recess being configured to receive the barrel of the syringe.

### Example 33

The apparatus of any of Examples 24 through 32, wherein the main body portion further includes at least one distal recess disposed distal of the proximal channel, the at least one distal recess being configured to receive at least one finger flange of the syringe.

### Example 34

The apparatus of any of Examples 24 through 33, further comprising first and second detents extending laterally inwardly relative to the longitudinal axis from the first and second beams, respectively, the first and second detents being configured to frictionally engage the barrel of the syringe.

### Example 35

The apparatus of any of Examples 24 through 34, further comprising first and second detents extending laterally inwardly relative to the longitudinal axis from the first and second flanges, respectively, the first and second detents being configured to frictionally engage the barrel of the syringe.

### Example 36

The apparatus of any of Examples 24 through 35, wherein the first and second beams are configured to deflect laterally away from each other from respective undeflected states toward respective deflected states, wherein the first and beams are resiliently biased toward the respective undeflected states.

### Example 37

The apparatus of any of Examples 24 through 36, wherein the first and second flanges are configured to deflect laterally away from each other from respective undeflected states toward respective deflected states, wherein the first and beams are resiliently biased toward the respective undeflected states.

### Example 38

The apparatus of any of Examples 24 through 37, further comprising a syringe, the syringe comprising: (i) a barrel having a second stop surface disposed distal of the first stop surface, and (ii) a plunger slidably disposed in the barrel and configured to selectively engage each of the first and second stop surfaces.

### Example 39

The apparatus of Example 38, wherein the plunger is slidably received by the proximal channel.

### Example 40

The apparatus of any of Examples 38 through 39, wherein the barrel is securely gripped by the first and second beams.

### Example 41

The apparatus of any of Examples 38 through 40, wherein the barrel is securely gripped by the first and second flanges.

### Example 42

The apparatus of any of Examples 38 through 41, further comprising a delivery instrument, the delivery instrument comprising: (i) a flexible cannula configured to fit between a choroid and a sclera in a patient's eye, and (ii) a needle slidably disposed in the cannula, the needle defining a lumen in fluid communication with the barrel of the syringe.

### Example 43

The apparatus of Example 42, wherein the plunger is configured to drive approximately 100 microliters of fluid from the barrel to a distal tip of the needle.

### Example 44

A system, comprising: (a) a delivery instrument having a distal end; (b) a syringe including: (i) a barrel, and (ii) a plunger slidably disposed in the barrel; (c) a fluid path extending between the barrel and the distal end of the instrument; and (d) a dose dock including: (i) a receptacle configured to receive the barrel of the syringe, and (ii) a stop surface disposed at a fixed location proximal of the receptacle, the stop surface facing proximally, wherein the plunger is configured to be advanced distally relative to the barrel from a first longitudinal position to a second longitudinal position to thereby purge any air from the fluid path, the stop surface being configured to selectively engage the plunger of the syringe to prevent distal advancement of the plunger relative to the barrel beyond the second longitudinal position.

### Example 45

A method of operating a system, the system including (i) a dose dock including (A) a receptacle, and (B) a first stop surface, and (ii) a syringe including (A) a barrel having a second stop surface, and (B) a plunger slidably disposed in the barrel, the method comprising: (a) retracting the plunger proximally relative to the barrel to a first longitudinal position to thereby draw fluid from a fluid source into the barrel; (b) inserting the barrel of the syringe into the receptacle of the dose dock; (c) decoupling the fluid source from the barrel; (d) coupling the barrel to an instrument; (e) with the barrel of the syringe disposed in the receptacle of the dose dock and coupled to the instrument, advancing the plunger distally relative to the barrel to a second longitudinal position to thereby purge any air from a fluid path between the barrel and a distal end of the instrument, the plunger engaging the first stop surface in the second longitudinal position, the first stop surface preventing further distal advancement of the plunger relative to the barrel; (f) removing the barrel of the syringe from the receptacle of the dose dock; and (g) with the barrel of the syringe removed from the receptacle of the dose dock and coupled to the instrument, advancing the plunger distally relative to the barrel to a third longitudinal position to thereby dispense the fluid from the barrel through the distal end of the instrument, the plunger engaging the second stop surface in the third longitudinal position, the second stop surface preventing further distal advancement of the plunger relative to the barrel.

### VII. MISCELLANEOUS

To the extent that several examples herein are described in the context of a cannula guide being positioned near an already-formed scleral incision (23), it should be understood that other kinds of procedures may be employed. For instance, in some variations of the procedures described herein, the cannula guide may be secured to the eye (20) first; and then the scleral incision (23) may be formed after the cannula guide has been secured to the eye (20). Other suitable steps and sequences that may be carried out in procedures that include a combination of a scleral incision (23) and a cannula guide will be apparent to those skilled in the art in view of the teachings herein.

It should be understood that any of the versions of the instruments described herein may include various other features in addition to or in lieu of those described above. By way of example only, any of the devices herein may also include one or more of the various features disclosed in any of the various references that are incorporated by reference herein.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by an operator immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1-24. (canceled)

25. An apparatus, comprising:
(a) a main body portion including:
(i) a proximal channel extending along a longitudinal axis and configured to slidably receive a plunger of a syringe, and
(ii) a first stop surface disposed at a fixed location proximal of the proximal channel, the first stop surface facing proximally and being configured to selectively engage the plunger of the syringe to arrest distal advancement of the plunger relative to a barrel of the syringe;
(b) first and second deflectable beams disposed distal of the proximal channel and on first and second sides of the longitudinal axis, respectively, the first and second beams being configured to cooperate with each other to securely grip the barrel of the syringe; and
(c) first and second deflectable flanges disposed distal of the first and second beams on the first and second sides of the longitudinal axis, respectively, the first and second flanges being configured to cooperate with each other to grip the barrel of the syringe.

26. The apparatus of claim 25, further comprising a hilt portion disposed distal of the main body portion, the hilt portion including at least one finger flange extending laterally outwardly relative to the longitudinal axis and configured to be gripped by a user.

27. The apparatus of claim 26, wherein the hilt portion includes the first and second beams.

28. The apparatus of claim 26, further comprising a support portion disposed distal of the hilt portion, the support portion including an elongate arm and a cradle at a distal end of the elongate arm.

29. The apparatus of claim 28, wherein the support portion includes the first and second flanges.

30. The apparatus of claim 28, wherein the cradle includes a distal channel extending along the longitudinal axis and configured to receive the barrel of the syringe.

31. The apparatus of claim 25, wherein the main body portion further includes a proximal recess disposed proximal of the proximal channel to define the first stop surface.

32. The apparatus of claim 25, wherein the main body portion further includes an intermediate recess disposed distal of the proximal channel, the intermediate recess being configured to receive the barrel of the syringe.

33. The apparatus of claim 25, wherein the main body portion further includes at least one distal recess disposed distal of the proximal channel, the at least one distal recess being configured to receive at least one finger flange of the syringe.

34. The apparatus of claim 25, further comprising first and second detents extending laterally inwardly relative to the longitudinal axis from the first and second beams, respectively, the first and second detents being configured to frictionally engage the barrel of the syringe.

35. The apparatus of claim 25, further comprising first and second detents extending laterally inwardly relative to the longitudinal axis from the first and second flanges, respectively, the first and second detents being configured to frictionally engage the barrel of the syringe.

36. The apparatus of claim 25, wherein the first and second beams are configured to deflect laterally away from each other from respective undeflected states toward respective deflected states, wherein the first and beams are resiliently biased toward the respective undeflected states.

37. The apparatus of claim 25, wherein the first and second flanges are configured to deflect laterally away from each other from respective undeflected states toward respective deflected states, wherein the first and beams are resiliently biased toward the respective undeflected states.

38. The apparatus of claim 25, further comprising a syringe, the syringe comprising:
(i) a barrel having a second stop surface disposed distal of the first stop surface, and
(ii) a plunger slidably disposed in the barrel and configured to selectively engage each of the first and second stop surfaces.

39. The apparatus of claim 38, wherein the plunger is slidably received by the proximal channel.

40. The apparatus of claim 38, wherein the barrel is securely gripped by the first and second beams.

41. The apparatus of claim 38, wherein the barrel is securely gripped by the first and second flanges.

42. The apparatus of claim 38, further comprising a delivery instrument, the delivery instrument comprising:
(i) a flexible cannula configured to fit between a choroid and a sclera in a patient's eye, and
(ii) a needle slidably disposed in the cannula, the needle defining a lumen in fluid communication with the barrel of the syringe.

43. (canceled)

44. A system, comprising:
(a) a delivery instrument having a distal end;
(b) a syringe including:
(i) a barrel, and
(ii) a plunger slidably disposed in the barrel;
(c) a fluid path extending between the barrel and the distal end of the instrument; and
(d) a dose dock including:
(i) a receptacle configured to receive the barrel of the syringe, and
(ii) a stop surface disposed at a fixed location proximal of the receptacle, the stop surface facing proximally,
wherein the plunger is configured to be advanced distally relative to the barrel from a first longitudinal position to a second longitudinal position to thereby purge any air from the fluid path, the stop surface being configured to selectively engage the plunger of the syringe to prevent distal advancement of the plunger relative to the barrel beyond the second longitudinal position.

45. A method of operating a system, the system including (i) a dose dock including (A) a receptacle, and (B) a first stop surface, and (ii) a syringe including (A) a barrel having a second stop surface, and (B) a plunger slidably disposed in the barrel, the method comprising:
(a) retracting the plunger proximally relative to the barrel to a first longitudinal position to thereby draw fluid from a fluid source into the barrel;
(b) inserting the barrel of the syringe into the receptacle of the dose dock;
(c) decoupling the fluid source from the barrel;
(d) coupling the barrel to an instrument;
(e) with the barrel of the syringe disposed in the receptacle of the dose dock and coupled to the instrument, advancing the plunger distally relative to the barrel to a second longitudinal position to thereby purge any air from a fluid path between the barrel and a distal end of the instrument, the plunger engaging the first stop surface in the second longitudinal position, the first stop surface preventing further distal advancement of the plunger relative to the barrel;
(f) removing the barrel of the syringe from the receptacle of the dose dock; and
(g) with the barrel of the syringe removed from the receptacle of the dose dock and coupled to the instrument, advancing the plunger distally relative to the barrel to a third longitudinal position to thereby dispense the fluid from the barrel through the distal end of the instrument, the plunger engaging the second stop surface in the third longitudinal position, the second stop surface preventing further distal advancement of the plunger relative to the barrel.
